(19) United States
(12) Patent Application Publication
(10) Pub. No.: US 2025/0163407 A1
(43) Pub. Date: May 22, 2025

(54) METHODS SELECTIVELY DEPLETING NUCLEIC ACID USING RNASE H

(71) Applicant: Jumpcode Genomics, Inc., San Diego, CA (US)

(72) Inventor: Keith BROWN, Carlsbad, CA (US)

(21) Appl. No.: 18/834,794

(22) PCT Filed: Feb. 2, 2023
(86) PCT No.: PCT/US2023/061880

§ 371 Date: Jul. 31, 2024

Related U.S. Application Data
(60) Provisional application No. 63/306,934, filed on Feb. 4, 2022.

(51) Int. Cl. C12N 15/10; C12Q 1/44; C12Q 1/6806; C12Q 1/6809; C12Q 1/6811; C12Q 1/6813

(52) U.S. Cl. CPC C12N 15/1093; C12Q 1/44; C12Q 1/6806; C12Q 1/6809; C12Q 1/6811; C12Q 1/6813; G01N 2333/922

(57) ABSTRACT

Provided herein are methods and compositions for a simplified and cost effective method for removing unwanted nucleic acids from a sample using RNase H.

### CROSS-REFERENCE

This application claims the benefit of U.S. Provisional Application No. 63/306,934, filed Feb. 4, 2022, which is incorporated herein by reference in its entirety.

### BACKGROUND

The disclosure herein relates to the field of molecular biology, such as methods and compositions for detecting, enriching and/or altering a target nucleic acid in a sample. The methods and compositions are applicable to biological, clinical, forensic, and environmental samples.

Efficient removal of one DNA species from a sample containing multiple species of DNA is desired for applications such as microbiome analysis and infectious disease monitoring. In addition, simply cleaning up contaminants for synthetic biology or other applications is desired. To date, methods such as differential lysis followed by DNAse digestion of the cells that were easier to lyse (i.e. human vs. bacterial) have proven to be costly and inefficient. Other methods rely on methylation and attempt to pull out methylated sequences (human) from non-methylated sequences (bacteria) by use of methylation specific nucleases or methylation specific DNA binding proteins/antibodies. Further, CRISPR based methods have been described. A cost-efficient, easy, and viable method of removing a guided selections of nucleic acid from a nucleic acid sample is therefore necessary.

### SUMMARY

In one aspect, provided herein is a method of enriching a target nucleic acid from a sample comprising a plurality of nucleic acid molecules, the method comprising depleting unwanted nucleic acid from the plurality of nucleic acid molecules of the sample by converting the target nucleic acids (or nucleic acid in the sample) into RNA, hybridization the RNA comprising the non-target nucleic acid, with single stranded DNA comprising unwanted nucleic acid comprising non-target nucleic acid, treating the hybridized product with RNase H, collecting the resultant undigested RNA thereby enriching for the nucleic acid comprising the target nucleic acid from the sample, optionally converting the resultant undigested RNA to DNA for suitable downstream use.

In one aspect, provided herein is a method of enriching a target nucleic acid from a sample comprising a plurality of nucleic acid molecules, the method comprising depleting unwanted polynucleotide sequence elements from the plurality of nucleic acid molecules of the sample by converting the nucleic acid from the sample in its entirety comprising the target nucleic acids into RNA, hybridization the RNA comprising the non-target nucleic acid with single stranded DNA generated from nucleic acid comprising unwanted nucleic acid, e.g., nucleic acid comprising non-target nucleic acid, treating the hybridized product with RNase H, collecting the resultant undigested RNA thereby enriching for the nucleic acid comprising the target nucleic acid from the sample, optionally converting the resultant undigested RNA to DNA for suitable downstream use.

In one aspect, provided herein is a method of enriching a target nucleic acid from a sample comprising a plurality of nucleic acid molecules, the method comprising depleting unwanted polynucleotide sequence elements from the plurality of nucleic acid molecules of the sample by converting the target nucleic acids (or the nucleic acid in the sample) into RNA, hybridizing the RNA comprising the non-target nucleic acid, with oligonucleotide DNA molecules comprising non-target nucleic acid sequence elements, treating the hybridized product with RNase H, collecting the resultant undigested RNA thereby enriching the nucleic acid comprising the target nucleic acid from the sample that comprised the plurality of nucleic acid molecules, optionally converting the resultant undigested RNA to DNA for suitable downstream use. In some embodiments, the sample comprising the plurality of nucleic acid molecules comprises sequence elements comprising target nucleic acid sequence element and unwanted or non-target sequence elements. In one embodiment, the sample is processed to generate RNA that comprises, e.g., the sequence elements comprising all the sequence elements in the sample; and simultaneously processing a portion or aliquot of the sample to generate DNA molecules comprising non-target nucleic acid sequence elements, prior to hybridizing. In some embodiments, the sample comprising the plurality of nucleic acid molecules comprises sequence elements comprising target nucleic acid sequence element and an abundance of unwanted or non-target sequence elements. Removal or depletion of the unwanted or non-target sequence elements using the method as described herein results in cleaner nucleic acid enriched in sequence elements comprising target nucleic acid sequence element.

In one aspect, provided herein is an efficient method of depleting unwanted nucleic acid from a sample comprising a plurality of nucleic acid molecules by using RNase H, the method comprising, tagmenting the genomic DNA by treating the genomic DNA from a sample with a transpososome complex that will fragment and attach transposons to the end of the double stranded DNA molecules. The transposition reaction tagments one of the DNA on each end of the tagmented fragment. In some embodiments, the tagmentation process is accompanied by addition of a promoter and/or adapter sequences at the fragmented end. In one embodiment, an additional step of filling in gaps between fragmented ends is required. This is accomplished either through PCR or polymerase extension from the 3′ end of the target template through the adapter sequence that was added on each end transposons. In some embodiments, addition of a promoter and/or adapter sequences at the fragmented end comprise addition of a T7 promoter sequence. In some embodiments, the addition of adapter sequences at the fragmented end comprises addition of additional synthetic oligonucleotide sequences including, but not limited to NGS adapter sequences, sample barcodes and unique molecule identifiers (UMIs). The method comprises, for example, use of T7 polymerase to amplify the genomic DNA to create single stranded RNA constructs with sample derived sequence flanked by the NGS adapter sequences. The method further comprises, for example, the step of incubating the single stranded RNA constructs with DNA probes (single stranded) that comprise unwanted sequences to be removed thereby generating RNA:DNA hybrid molecules; treating the resultant mixture comprising the RNA:DNA hybrid molecules with RNase H that digests the RNA in the RNA:DNA hybrid molecules; and collecting the resulting undigested RNA constructs, thereby depleting unwanted nucleic acid from the sample.

In some embodiments, the collected undigested RNA constructs can have a large number of downstream applications, for example, preparing cDNA comprising enriched sequence, depleted of the unwanted nucleic acid, preparing an enriched nucleic acid sample, preparing a barcoded library, preparing a probe library, preparing an adapter-ligated enriched cDNA library; detecting specific low frequency sequences in the enriched sequence or generating a therapeutic comprising a target sequence from the enriched sequence. In some embodiments, the method comprises conversion of the non-digested RNA comprising the target nucleic acid, free of or relatively enriched from the unwanted collected non-target nucleic acid, into DNA, using RT-PCR. In one embodiment, a cDNA is generated comprising the enriched nucleic acid, depleted of the non-target nucleic acid. In one embodiment, a double stranded DNA is generated from the undigested RNA comprising the enriched target sequence using methods known in the art, in order to generate double-stranded DNA library.

In one aspect, provided herein is a method of detecting the presence or absence of a target nucleic acid from a sample comprising a plurality of nucleic acid molecules, the method comprising depleting unwanted nucleic acid from the plurality of nucleic acid molecules by selective hybridization of an RNA comprising the non-target nucleic acid with single stranded DNA oligonucleotide molecules of a nucleic acid member of the plurality of nucleic acids, treating the hybridized product with RNase H and collecting the resulting undigested RNA, detecting the presence or absence of a target nucleic acid in the resulting undigested RNA or in a DNA derived thereof.

In one aspect, provided herein is a method of amplifying a target nucleic acid from a sample comprising a plurality of nucleic acid molecules, the method comprising depleting unwanted nucleic acid from the plurality of nucleic acid molecules by selective hybridization of an RNA comprising the non-target nucleic acid with single stranded DNA oligonucleotide molecules of a nucleic acid member of the plurality of nucleic acids, treating the hybridized product with RNase H and collecting the resulting undigested RNA, amplifying the target nucleic acid from the undigested RNA or a DNA derived thereof.

Provided herein is a method of selective depletion of non-target nucleic acid sequences from a sample comprising a first plurality of nucleic acid molecules comprising target nucleic acid sequence and a second plurality of nucleic acid molecules comprising non-target nucleic acid sequences, the method comprising: (a) generating RNA molecules from fragments generated from the first and second plurality of nucleic acid molecules, each fragment comprising one or more nucleic acid sequences additionally placed within the nucleic acid prior to or while generating the RNA, the one or more nucleic acid sequences comprising a promoter sequence for an RNA polymerase, and wherein the sum of the RNA molecules generated from the fragments comprise the plurality of nucleic acid molecules; (b) separately, generating DNA probes comprising non-target nucleic acid sequences; (c) hybridizing RNA molecules from (a) and DNA probes from (b) under conditions suitable for generating RNA:DNA hybrid molecules; (d) subjecting the RNA:DNA hybrid from (c) to RNase H treatment, thereby selectively depleting the RNA in the RNA:DNA hybrid; and recovering unhybridized RNA comprising the target nucleic acid sequence, selectively depleted of the non-target nucleic acid sequence.

In some embodiments, DNA probes, e.g. DNA oligonucleotide sequences comprising an unwanted nucleic acid of step (b) above, can be generated using various different methods known in the art, such using samples from a microarray, using short fragments of the an oligo pool used in microarray processes, for example. In one embodiment, the DNA probe may be generated from would a genomic DNA, fragments, sheared or digested into shorter oligonucleotides by methods known in the art. In one embodiments, the DNA probes can be suitably generated in a cost-effective manner, e.g., using a genomic DNA to generate oligonucleotide probes, provided the genomic DNA is the suitable source of the non-target nucleic acid. For example, the method as described in the preceding sentence could be useful when the target nucleic acid comprises a genomic DNA of a test organism, and the non-target nucleic acid is a contaminant DNA from a different organism; e.g. host DNA versus microbial DNA, or vice versa.

Provided herein is a method of detecting the presence or absence of a target nucleic acid from a sample comprising a first plurality of nucleic acid molecules comprising target nucleic acid sequence and a second plurality of nucleic acid molecules comprising non-target nucleic acid sequences, the method comprising: (a) depleting non-target nucleic acid sequences from the first and second plurality of nucleic acid molecules by selective hybridization of an RNA comprising the non-target nucleic acid with single stranded DNA oligonucleotide molecules of a nucleic acid member of the plurality of nucleic acids that comprise the target nucleic acid, under conditions suitable for generating RNA:DNA hybrid molecules; treating the nucleic acid comprising the generated RNA:DNA hybrid molecules with RNase H thereby digesting the RNA in the RNA:DNA hybrid molecules; and collecting the resulting undigested RNA; (b) detecting the presence or absence of a target nucleic acid sequence in the resulting undigested RNA or in a DNA derived therefrom. In some embodiments, the resulting undigested RNA is optionally passed through purification steps to remove unwanted nucleic acid fragments and digestion products. In some embodiments the purification is performed using filtrations and/or other size-based selection procedures. In some embodiments, commercial size exclusion cleaning kits are used for the cleaning up or purification process. In some embodiments, the purified RNA is converted to double stranded DNA by reverse transcriptase (RT) reactions, or RT-PCT amplifications. In some embodiments, the double stranded DNA is optionally cleaned/purified for end use or storage. In some embodiments, collecting the resulting undigested RNA comprises the optional cleaning step as described, or is replaced by the RT-PCR step that can use the adapter sequences in the undigested RNA to amplify the enriched nucleic acid comprising the target nucleic acid.

Provided herein is a method of enriching a target nucleic acid from a sample comprising a first plurality of nucleic acid molecules comprising target nucleic acid sequence and a second plurality of nucleic acid molecules comprising non-target nucleic acid sequences, the method comprising: depleting non-target nucleic acid sequences from the first and second plurality of nucleic acid molecules by selective hybridization of an RNA comprising a non-target nucleic acid with single stranded DNA oligonucleotide molecules of a nucleic acid member of the plurality of nucleic acids in the sample that comprises unwanted or non-target nucleic acid, under conditions suitable for generating RNA:DNA hybrid molecules; treating the nucleic acid comprising the generated RNA:DNA hybrid molecules with RNase H thereby digesting the RNA in the RNA:DNA hybrid molecules; and collecting the resulting undigested RNA; enriching the target nucleic acid in the resulting undigested RNA or in a DNA derived therefrom.

In various embodiments of methods provided herein, in some embodiments, the method further comprises forming a nucleic acid library comprising the target nucleic acid sequence. In some embodiments, the RNA comprising the target nucleic acid is generated by in vitro transcription of the plurality of nucleic acid molecules comprising target nucleic acid sequence and non-target nucleic acid sequences. In some embodiments, prior to generating the RNA, the plurality of nucleic acid molecules comprising target nucleic acid sequence and non-target nucleic acid sequences are fragmented by insertion of an RNA polymerase promoter sequence at intervals within the plurality of nucleic acid molecules, such that each fragment comprises an RNA polymerase promoter suitable for amplification of the sample, or suitable for filing in gaps between fragments. In some embodiments, the RNA polymerase promoter sequence comprises a sequence from a promoter selected from the list of promoters consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. In some embodiments, the promoter sequence is a T7 promoter sequence. In some embodiments, the method further comprises insertion of one or more synthetic adaptor nucleic acid sequences or one or more primer sequences. In some embodiments, the insertion is performed by a transposase. In some embodiment, the insertion comprises insertion of a transposon complex. In some embodiments, the transposase is a DNA transposase, such as Tn5 transposase, or a Mu transposase. In some embodiments, the transposon is an RNA transposon. In some embodiments the transposase is a retrotransposase. In some embodiments the retrotransposase is a non-LTR retrotransposase, e.g., a LINE1 element. In some embodiments, the transposase inserts a first insert sequence comprising a first transposon at a first insertion site, and a second insert sequence at a second insertion site comprising a second transposon on the genomic DNA. In some embodiments, the transposase inserts a first promoter sequence and/or a first adaptor sequence at the first insertion site; and a second promoter sequence and/or a second adaptor sequence at the second insertion site. In some embodiments, the transposase inserts a third or subsequent promoter sequence and/or a third or subsequent adaptor sequence at the third or subsequent insertion site. In some embodiments, a first transposase inserts a first transposon and/or a first promoter sequence and/or a first adaptor sequence at the first insertion site; and a second transposase inserts a second transposon and/or a promoter sequence and/or a second adaptor sequence at the second insertion site. In some embodiments, multiple transposase can act at multiple insertion sites thereby creating multiple insertion events across the plurality of nucleic acid molecules, wherein the multiple insertion events comprise an insertion of a transposon, a promoter sequence and/or an adaptor sequence, identical to or different from one another. In some embodiments, multiple different combinations of promoters or combinations of transposase enzymes with different characteristics such as AT or GC biased insertion sites can be employed in the process. In some embodiments, the first and the second insertion site is at least 250 nucleotides apart from the first insertion site. In some embodiments, the first and the third insertion site is at least 250 nucleotides apart from the first insertion site. In some embodiments, the first insert sequence comprises a restriction endonuclease cleavage site between said first primer binding site and said second primer binding site. In some embodiments, the RNA is generated by in vitro transcription from the inserted RNA polymerase promoter. In some embodiments, RNA generated by in vitro transcription results in a high fidelity copy of the plurality of nucleic acid molecules comprising target nucleic acid sequence and non-target nucleic acid sequences. In some embodiments, sample comprises heterogenous nucleic acid. In some embodiments, sample comprises genomic DNA of one or more species. In some embodiments, sample comprises genomic DNA of a microbial species comprising the target nucleic acid sequence, and genomic DNA of a host species comprising the non-target nucleic acid sequences. In some embodiments, sample comprises genomic DNA of a host species comprising the target nucleic acid sequence, and genomic DNA of a microbial species comprising the non-target nucleic acid sequences. In some embodiments, DNA probe is generated from nucleic acid comprising non-target nucleic acid sequences. In some embodiments, DNA probes are oligonucleotide probes that are less than 500 nucleotides long. In some embodiments, prior to hybridization, the nucleic acids are subjected to denaturing conditions in elevated temperatures of 60° C. or more for at least 10 minutes. In some embodiments, depleting comprises reducing at least by greater than 50% the sequence comprising the non-target nucleic acid sequences compared to the level prior to depletion.

In another aspect, there are provided methods of selective depletion of non-target nucleic acid sequences from a sample wherein the sample comprises a plurality of nucleic acid molecules comprising target nucleic acid sequence and non-target nucleic acid sequences. In some cases, the method comprises (a) generating RNA molecules from fragments generated from the plurality of nucleic acid molecules, each fragment comprising an insert comprising a promoter sequence for an RNA polymerase, wherein the sum of the RNA molecules generated from the fragments comprise the plurality of nucleic acid molecules. In some cases, the method comprises, (b) generating DNA probes from non-target nucleic acid sequences. In some cases, the method comprises (c) hybridizing RNA molecules from (a) and DNA probes from (b) under conditions suitable for generating RNA:DNA hybrid molecules. In some cases, the method comprises (d) subjecting the nucleic acids comprising the RNA:DNA hybrid from (c) to RNase H treatment, thereby selectively depleting the RNA in the RNA:DNA hybrid; and recovering unhybridized RNA comprising the target nucleic acid sequence, selectively depleted of the non-target nucleic acid sequence.

In a further aspect, there are provided, methods of detecting the presence or absence of a target nucleic acid from a sample comprising a plurality of ribonucleic acid (RNA) molecules comprising target RNA sequence and non-target RNA sequences. In some cases, the method comprises depleting non-target RNA sequences from the plurality of RNA molecules by selective hybridization of an RNA comprising the non-target RNA with single stranded DNA oligonucleotide molecules having sequence identity to the non-target RNA under conditions suitable for generating RNA:DNA hybrid molecules; treating the nucleic acid comprising the generated RNA:DNA hybrid molecules with RNase H thereby digesting the RNA in the RNA:DNA hybrid molecules; and collecting the resulting undigested RNA. In some cases, the method comprises detecting the presence or absence of a target nucleic acid sequence in the resulting undigested RNA or in a DNA derived therefrom.

In another aspect, there are provided methods of enriching a target ribonucleic acid (RNA) from a sample comprising a plurality of RNA molecules comprising target RNA sequence and non-target RNA sequence. In some cases, the method comprises depleting the non-target RNA sequences from the plurality of RNA molecules by selective hybridization of an RNA comprising the non-target sequence with a single stranded DNA oligonucleotide molecule having sequence identity with the non-target sequence under conditions suitable for generating RNA:DNA hybrid molecules; treating the nucleic acid comprising the generated RNA:DNA hybrid molecules with RNase H thereby digesting the RNA in the RNA:DNA hybrid molecules; and collecting the resulting undigested RNA. In some cases, the method comprises enriching the target nucleic acid in the resulting undigested RNA or in a DNA derived therefrom.

In various aspects of methods provided herein, in some cases the method further comprises forming a nucleic acid library comprising the target nucleic acid sequence. In some cases, the sample comprises heterogenous nucleic acid. In some cases, the sample comprises RNA derived from one or more species. In some cases, the sample comprises RNA derived from one or more different organisms. In some cases, the sample comprises RNA derived from a microbial species comprising the target nucleic acid sequence, and RNA derived from a host species comprising the non-target nucleic acid sequences. In some cases, the sample comprises RNA derived from a host species comprising the target nucleic acid sequence, and RNA derived from a microbial species comprising the non-target nucleic acid sequences. In some cases, the DNA probe is generated from nucleic acid comprising non-target nucleic acid sequences. In some cases, the DNA probe is synthesized. In some cases, the DNA probe is generated from a sample nucleic acid or a portion thereof, by cleavage to generate oligonucleotide fragments used as probes. In some cases, the DNA probes are oligonucleotide probes that are less than 500 nucleotides long. In some cases, the prior to hybridization, the nucleic acids are subjected to denaturing conditions in elevated temperatures of 60° C. or more for at least 10 minutes. In some cases, depleting comprise reducing at least by greater than 50% the sequence comprising the non-target nucleic acid sequences compared to the level prior to depletion.

In another aspect, provided herein is a nucleic acid molecule comprising a sequence comprising a target nucleic acid sequence that is enriched from a sample comprising a plurality of nucleic acid molecules comprising target nucleic acid sequence and non-target nucleic acid sequences using any one of the methods described herein.

### BRIEF DESCRIPTION OF FIGURES

FIG. 1A depicts a generalized exemplary schematic representative of the workflow describing a method to generate depletion of unwanted nucleic acid from a sample, using RNase H.

FIG. 1B illustrates an index of each graphic representation of nucleic acid structure depictions in the representative schematic workflow of FIG. 1A and elsewhere in the figures.

FIG. 2 depicts graphically simplified workflow representing a low-cost production of DNA probes described in the method steps represented in the schematic workflow of FIG. 1A and elsewhere in the specification.

FIG. 3 is a representative data depicting transposon insertion frequency in a method represented in the schematic workflow of FIG. 1A. The data shows that the transposon end with single-stranded T7 promoter inserts into genomic DNA at an apparent average frequency of approximately 1 insertion in 1000 bp, thereby causing DNA fragmentation. Tn, transposon; Tnp, transposase; nt, nucleotides; ss, single stranded; ds, double stranded.

FIG. 4 is a representative data showing in vitro transcription results from T7 promoters, in the method represented in the schematic workflow FIG. 1A, in which T7 promoter-containing transposons are inserted into a genomic DNA (gDNA) sample.

### DETAILED DESCRIPTION

Methods and compositions disclosed herein are based on a simplified and cost effective improvement encompassing using RNase H to remove unwanted nucleic acid from a sample in a step that would otherwise require multi-step, expensive, and/or otherwise precision sequence-specific nucleic acid removal methods while at the same time achieving a precise removal of unwanted nucleic acid sequences from the sample, and enrichment of nucleic acid depleted of unwanted sequences that constitute noise. The improvement replaces at least a use of sequence specific excision by specific endonuclease and other enzymes. The methods can be applicable to or adopted into a large variety of applications and can be benefit a large number of downstream usages. For example, the methods herein can be used to deplete unwanted DNA sequences from a sample as well as to deplete unwanted RNA sequences from a sample.

In an aspect, the methods described herein comprise, first, obtaining synthetic sequence tagged RNA from a genomic DNA in a sample comprising unwanted DNA, comprising transposase mediated insertion (transpososome tagmenting) of a promoter sequence for a polymerase mediated transcription; additionally inserting synthetic adapter sequences, tags or UMI etc at multiple sites in the genomic DNA followed by obtaining a transcript RNA sequence of the tagmented DNA by subjecting the tagmented DNA to polymerase reaction; separately, preparing oligonucleotide DNA probes from a nucleic acid comprising unwanted DNA; contacting the RNA obtained by the polymerase reaction with the oligonucleotide DNA probes, thereby obtaining a nucleic acid mixture comprising DNA:RNA hybridized products and unhybridized nucleic acids; subjecting the nucleic acid mixture to RNase H in conditions that digests RNA in the RNA:DNA hybridized products; collecting the resulting undigested RNA thereby depleted of the unwanted sequences. The resultant RNA can be converted to double stranded DNA library. The method is adaptable to downstream diagnostic and therapeutic applications.

For example, a nucleic acid isolated from a sample comprising a microbial nucleic acid in a host genomic DNA wherein the microbial nucleic acid comprises a sequence of interest. For identification and/or isolation of a sequence in the microbial nucleic acid, a bulk of the host genomic DNA can be removed (depleted), using the methods described herein.

In another aspect, there are provided methods comprising obtaining an RNA from a sample comprising unwanted RNA. In this method, DNA probes having a sequence with identity to the unwanted sequence are added to the RNA sample under conditions that promote hybridization of the DNA probe with the unwanted RNA. RNase H can then be added to the sample where it can digest the unwanted RNA from the sample. In some embodiments, adapters are added to the RNA in the sample by ligating adapters to the 5′ and 3′ ends of the RNA molecules in the sample. RNA adapters can be ligated to RNA in the sample by using an RNA ligase, such as T4 RNA ligase. The adapter-ligated RNA can be hybridized with DNA probes that bind to the unwanted sequence and then treated with RNase H to digest the unwanted RNA. In some cases, the undigested RNA molecules are amplified using adapter-specific RT-PCR.

Alternatively, in another aspect, for example, a bulk of a microbial sample contaminating a host sample can be removed or depleted using the method described above.

For example, a nucleic acid isolated from a sample comprising a sequence of interest (e.g. a target sequence for diagnostic purpose; or a target sequence for amplification and use for a library preparation), is present in a sensitive amount or proportion with respect to sequences that are not of interest in the isolated nucleic acid; a large proportion of nucleic acid comprising unwanted sequences can be removed (removed or interchangeably depleted, interchangeably) using the method described herein. The isolated nucleic acid sample could be, for example, genomic DNA, and the depleted sequence could be ribosomal DNA, or repetitive DNA, thereby enriching the sequence of interest which does not (in this example) lie within the ribosomal DNA sequence. In some embodiments, the starting material could be a cDNA sample. In some embodiments, the starting material could be a RNA sample.

For example, a nucleic acid sample comprises contaminated sequence from a heterogenous source, e.g., nucleic acid sample comprising multiple contaminant species, nucleic acid sample comprising multiple contaminant individuals, e.g. in forensic samples, or nucleic acid sample comprising multiple contaminant tissues of an individual of a species, and/or wherein a sequence of interest is present in a relatively low proportion to the unwanted contaminating sequences, the unwanted sequences can be removed using the methods described herein. Another example could be that the target nucleic acid is comprised in fetal DNA present in a biological sample of the mother.

In one embodiment, one of the advantages of using the method is that the method can achieve a sequence specific removal of nucleic acid from a sample, without having to use sequence specific, expensive and time consuming reagents and processes. For example, a genomic DNA from a first species contaminated with a genomic nucleic acid from a second or subsequent species, removal of the second or subsequent genomic nucleic acids is achieved using the methods described herein by generating DNA probes from the second or subsequent genomic nucleic acids hybridizing the probes with RNA generated from the sample, as described, and isolating the unhybridized RNA. A wide range of organisms are suitable sources for genomic DNA, such as eukaryotic, eubacterial or archaeal sources. Non-limiting sample sources include animal DNA, plant DNA, or generally, DNA is isolated from a biological sample such as a blood sample, a bodily fluid sample, a hair sample, a skin sample, a saliva sample, etc., as indicated elsewhere herein and as contemplated by one of skill in the art.

Non-limiting exemplary situations are provided herein for utilization of the technology described herein.

For example, a target sequence could be a vector sequence or a plasmid sequence or other sequences comprising a recombinant or synthetic nucleic acid, and the non-target unwanted nucleic acid is the nucleic acid of the host organism, e.g., a host bacteria in which the plasmid, or vector or other sequences comprising a recombinant or synthetic nucleic acid is amplified.

For example, ribosomal nucleic acid is an unwanted sequence that is contaminating a target sequence present in sensitive amount can be depleted from an isolated genomic DNA can be selectively removed as discussed above.

In one embodiment, one of the advantages of using the method can be highly adaptive, in that it can be adapted to any usage and requirement for depletion of a nucleic acid sequence. For example, unwanted DNA can be removed by highly selectively sequence specific process, contrary to the above, by using sequence directed guide RNA to generate DNA oligonucleotide probes for the method described above.

### Definitions

A partial list of relevant definitions is as follows.

“Amplified nucleic acid” or “amplified polynucleotide” can be any nucleic acid or polynucleotide molecule whose amount has been increased at least two fold by any nucleic acid amplification or replication method performed in vitro as compared to its starting amount. For example, an amplified nucleic acid can be obtained from a polymerase chain reaction (PCR) which amplifies DNA in an exponential manner (for example, amplification to 2n copies in n cycles). Amplified nucleic acid can also be obtained from a linear amplification.

“Amplification product” can refer to a product resulting from an amplification reaction such as a polymerase chain reaction.

An “amplicon” can be a polynucleotide or nucleic acid that is the source and/or product of natural or artificial amplification or replication events.

The term “biological sample” or “sample” generally refers to a sample that is generated de novo or that is wholly or in part isolated from a biological entity. The biological sample may show the nature of the whole of the biological entity from which it is obtained. Examples include, without limitation, bodily fluids, dissociated tumor specimens, cultured cells, and any combination thereof. Biological samples can come from one or more individuals. One or more biological samples can come from the same individual. One non limiting example would be if one sample came from an individual's blood and a second sample came from an individual's tumor biopsy. Examples of biological samples can include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. The samples may include nasopharyngeal wash. Examples of tissue samples of the subject may include but are not limited to, connective tissue, muscle tissue, nervous tissue, epithelial tissue, cartilage, cancerous or tumor sample, or bone. The sample may be provided from a human or animal. The sample may be provided from a mammal, including vertebrates, such as murines, simians, humans, farm animals, sport animals, or pets. The sample may be collected from a living or dead subject. The sample may be collected fresh from a subject or may have undergone some form of pre-processing, storage, or transport.

Nucleic acid sample as used herein refers to a nucleic acid sample for which sequence information is to be determined. A nucleic acid sample may be extracted from a biological sample above. Alternatively, a nucleic acid sample can be artificially synthesized, synthetic, or de novo synthesized. Often, the DNA sample is genomic, while in alternate cases the DNA sample is derived from a reverse-transcribed RNA sample. Some genomic samples, such as of viruses, are quite small. However, as used herein, a ‘genomic’ sample is often used in reference to a free-living organism's genome, such as a human, plant such as an agricultural crop, or even a human or plant or animal pathogen. In general. Such genomic samples comprise substantially large amounts of genomic information, such that amplification of a fraction of such sample such as 50% or greater of such a sample comprises amplification of a substantially large amount of sequence information. A nucleic acid sample can be a RNA sample that can be used directly in methods herein.

“Bodily fluid” generally can describe a fluid or secretion originating from the body of a subject. Bodily fluids can be a mixture of more than one type of bodily fluid mixed together. Some non-limiting examples of bodily fluids can be: blood, urine, bone marrow, spinal fluid, pleural fluid, lymphatic fluid, amniotic fluid, ascites, sputum, or a combination thereof.

“Complementary” or “complementarity,” or “reverse-complementarity” can refer to nucleic acid molecules that are related by base-pairing. Complementary nucleotides are, generally, A and T (or A and U), or C and G (or G and U). Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Hybridization temperatures are generally at least about 2° C. to about 6° C. lower than melting temperatures (Tm).

A “barcode” or “molecular barcode” is any sequence information used to label or identify adjacent nucleic acid molecule sequence. The barcode can label a molecule such as a nucleic acid or a polypeptide. The material for labeling can be associated with information. A barcode can be called a sequence identifier (i.e. a sequence-based barcode or sequence index). A barcode can be a particular nucleotide sequence, or a particular insertion pattern, for example within a repetitive region of a genome. A barcode can be used as an identifier. A barcode can be a different size molecule or different ending points of the same molecule. Barcodes can include a specific sequence within the molecule and a different ending sequence. For example, a molecule that is amplified from the same primer and has 25 nucleotide positions is different than a molecule that is amplified and has 27 nucleotide positions. The addition positions in the 27mer sequence can be considered a barcode. A barcode can be incorporated into a polynucleotide. A barcode can be incorporated into a polynucleotide by many methods. Some non-limiting methods for incorporating a barcode can include molecular biology methods. Some non-limiting examples of molecular biology methods to incorporate a barcode are through primers (e.g., tailed primer elongation), probes (i.e., elongation with ligation to a probe), or ligation (i.e., ligation of known sequence to a molecule).

A barcode can be incorporated into any region of a polynucleotide. The region can be known. The region can be unknown. The barcode can be added to any position along the polynucleotide. The barcode can be added to the 5′ end of a polynucleotide. The barcode can be added to the 3′ end of the polynucleotide. The barcode can be added in between the 5′ and 3′ end of a polynucleotide. A barcode can be added with one or more other known sequences. One non-limiting example is the addition of a barcode with a sequence adapter.

Barcodes can be associated with information. Some non-limiting examples of the type of information a barcode can be associated with information include: the source of a sample; the orientation of a sample; the region or container a sample was processed in; the adjacent polynucleotide; or any combination thereof.

Barcodes can be made from combinations of sequences (different from combinatorial barcoding) and can be used to identify a sample or a genomic coordinate and a different template molecule or single strand the molecular label and copy of the strand was obtained from. A sample identifier, a genomic coordinate and a specific label for each biological molecule may be amplified together. Barcodes, synthetic codes, or label information can also be obtained from the sequence context of the code (allowing for errors or error correcting), the length of the code, the orientation of the code, the position of the code within the molecule, and in combination with other natural or synthetic codes.

Incorporation of a barcode into a nucleic acid molecule indicates that the nucleic acid was present in a given sample at a given time period. Contiguous adjacent nucleic acid sequence sharing a common barcode or a common bar code pair is inferred to have been derived from a common molecule, particularly if the sample is diluted to less than an average of 2×, 1.5×, 1×, 0.7×, 0.5×, or 0.3× haploid genomes prior to barcode introduction.

Barcodes can be added before pooling of samples. When the sequences are determined of the pooled samples, the barcode can be sequenced along with the rest of the polynucleotide. The barcode can be used to associate the sequenced fragment with the source of the sample.

Barcodes can also be used to identify the strandedness sample. One or more barcodes can be used together. Two or more barcodes can be adjacent to one another, not adjacent to one another, or any combination thereof. Adapter orientation is often used to determine strandedness. For example, if an “A” adapter is always in the 5′-3′ direction in a first primer extension reaction, then one can infer the read starting from the A adapter would be the compliment of the strand that was initially primed.

Barcodes can be used for combinatorial labeling.

As indicated herein, standard single-letter amino acid residue abbreviations as known in the art are used to refer to the twenty amino acids involved in cellular ribosomally driven polypeptide synthesis. Thus, “L372P” for example, refers to a Leucine to Proline missense mutation at position 372 of a polypeptide.

“Combinatorial labeling” is a method by which two or more barcodes are used to label. The two or more barcodes can label a polynucleotide. The barcodes, each, alone can be associated with information. The combination of the barcodes together can be associated with information. A combination of barcodes can be used together to determine in a randomly amplified molecule that the amplification occurred from the original sample template and not a synthetic copy of that template. The length of one barcode in combination with the sequence of another barcode can be used to label a polynucleotide. The length of one barcode in combination with the orientation of another barcode can be used to label a polynucleotide. In other cases, the sequence of one barcode can be used with the orientation of another barcode to label a polynucleotide. The sequence of a first and a second bar code, in combination with the distance in nucleotides between them, is used to label or to identify a polynucleotide. The sequence of a first and a second bar code, in combination with the distance in nucleotides between them and the identity of the nucleotides between them, is used to label or to identify a polynucleotide.

“Degenerate” can refer to a nucleic acid or nucleic acid region that is comprised of random bases, for example as determined by comparison to other constituents of a population sharing other common characteristics. The terms “degenerate” and “random” can be used interchangeably when referring to nucleic acid sequences (e.g., “degenerate primers” or “random primers” or “degenerate probes” or “random probes”). The degenerate region can be of variable length. The degenerate region can comprise some portion of the whole nucleic acid (e.g., a semi-degenerate primer). The degenerate region can comprise the whole nucleic acid (e.g., a “degenerate primer”). A degenerate nucleic acid mix, or semi-degenerate nucleic acid mix may be comprised of every possible combination of base pairs, less than every possible combination of base pairs, or some combination of base pairs, a few combinations of base pairs, or a single base pair combination. A degenerate primer mix, or semi-degenerate primer mix can comprise mixes of similar but not identical primers.

“Double-stranded” can refer to two polynucleotide strands that have annealed through complementary base-pairing, such as in a reverse-complementary orientation.

“Known oligonucleotide sequence” or “known oligonucleotide” or “known sequence” can refer to a nucleic acid fragment such as a polynucleotide or longer nucleic acid sample molecule having a total or partial sequence that is known. A known oligonucleotide sequence can correspond to an oligonucleotide that has been designed, e.g., a universal primer for next generation sequencing platforms (e.g., Illumina, 454), a probe, an adaptor, a tag, a primer, a molecular barcode sequence, an identifier. A known sequence can comprise part of a primer. A known oligonucleotide sequence may not actually be known by a particular user but can be constructively known, for example, by being stored as data which may be accessible by a computer. A known sequence may also be a trade secret that is actually unknown or a secret to one or more users but may be known by the entity who has designed a particular component of the experiment, kit, apparatus or software that the user is using.

“Library” can refer to a collection of nucleic acids. A library can contain one or more target fragments. The target fragments can be amplified nucleic acids. In other instances, the target fragments can be nucleic acid that is not amplified. A library can contain nucleic acid that has one or more known oligonucleotide sequence(s) added to the 3′ end, the 5′ end or both the 3′ and 5′ end. The library may be prepared so that the fragments can contain a known oligonucleotide sequence that identifies the source of the library (e.g., a molecular identification barcode identifying a patient or DNA source). Two or more libraries can be pooled to create a library pool. Libraries may also be generated with other kits and techniques such as transposon mediated labeling, or “tagmentation” as known in the art. Kits may be commercially available, such as the Illumina NEXTERA kit (Illumina, San Diego, CA).

“Locus specific” or “loci specific” can refer to one or more loci corresponding to a location in a nucleic acid molecule (e.g., a location within a chromosome or genome). Loci can be associated with genotype. Loci may be directly isolated and enriched from the sample, e.g., based on hybridization and/or other sequence-based techniques, or they may be selectively amplified using the sample as a template prior to detection of the sequence. Loci may be selected on the basis of DNA level variation between individuals, based upon specificity for a particular chromosome, based on CG content and/or required amplification conditions of the selected loci, or other characteristics that will be apparent to one skilled in the art upon reading the present disclosure. A locus may also refer to a specific genomic coordinate or location in a genome as denoted by the reference sequence of that genome.

“Long nucleic acid” can refer to a polynucleotide of at least 1, 2, 3, 4, 5, 6, 7, 8, 9, or 10 kilobases or longer.

The term “melting temperature” or “Tm” commonly refers to the temperature at which a population of double-stranded nucleic acid molecules becomes half dissociated into single strands. Equations for calculating the Tm of nucleic acids are well known in the art. One equation that gives a simple estimate of the Tm value is as follows: Tm=81.5+16.6(log 10[Na+])0.41(%[G+C])−675/n−1.0 m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references can include more sophisticated computations, which take structural as well as sequence characteristics into account for the calculation of Tm.

“Nucleotide” can refer to a base-sugar-phosphate combination. Nucleotides are monomeric units of a nucleic acid sequence (e.g., DNA and RNA). The term nucleotide includes naturally and non-naturally occurring ribonucleoside triphosphates ATP, TTP, UTP, CTG, GTP, and ITP, for example and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and, for example, nucleotide derivatives that confer nuclease resistance on the nucleic acid molecule containing them. The term nucleotide as used herein also refers to dideoxyribonucleoside triphosphates (ddNTPs) and their derivatives. Illustrative examples of dideoxyribonucleoside triphosphates include, ddATP, ddCTP, ddGTP, ddITP, ddUTP and ddTTP, for example.

“Polymerase” can refer to an enzyme that links individual nucleotides together into a strand, using another strand as a template.

“Polymerase chain reaction” or “PCR” can refer to a technique for replicating a specific piece of selected DNA in vitro, even in the presence of excess non-specific DNA. Primers are added to the selected DNA, where the primers initiate the copying of the selected DNA using nucleotides and, typically, Taq polymerase or the like. By cycling the temperature, the selected DNA is repetitively denatured and copied. A single copy of the selected DNA, even if mixed in with other, random DNA, can be amplified to obtain thousands, millions, or billions of replicates. The polymerase chain reaction can be used to detect and measure very small amounts of DNA and to create customized pieces of DNA.

The term “polynucleotides” or “nucleic acids” may include but is not limited to various DNA, RNA molecules, derivatives, or combination thereof. These may include species such as dNTPs, ddNTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA.

A “primer” generally refers to an oligonucleotide used to, e.g., prime nucleotide extension, ligation and/or synthesis, such as in the synthesis step of the polymerase chain reaction or in the primer extension techniques used in certain sequencing reactions. A primer may also be used in hybridization techniques as a means to provide complementarity of a locus to a capture oligonucleotide for detection of a specific nucleic acid region.

“Primer extension product” can refer to the product resulting from a primer extension reaction using a contiguous polynucleotide as a template, and a complementary or partially complementary primer to the contiguous sequence.

“Sequencing,” “sequence determination,” and the like generally refers to any and all biochemical methods that may be used to determine the order of nucleotide bases in a nucleic acid.

A “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

The term “biotin,” as used herein, is intended to refer to biotin (5-[(3aS,4S,6aR)-2-oxohexahydro-1H-thieno[3,4-d]imidazol-4-yl]pentanoic acid) and any biotin derivatives and analogs. Such derivatives and analogs are substances which form a complex with the biotin binding pocket of native or modified streptavidin or avidin. Such compounds include, for example, iminobiotin, desthiobiotin and streptavidin affinity peptides, and also include biotin-.epsilon.-N-lysine, biocytin hydrazide, amino or sulfhydryl derivatives of 2-iminobiotin and biotinyl-ε-aminocaproic acid-N-hydroxysuccinimide ester, sulfo-succinimide-iminobiotin, biotinbromoacetylhydrazide, p-diazobenzoyl biocytin, 3-(N-maleimidopropionyl) biocytin. “Streptavidin” can refer to a protein or peptide that can bind to biotin and can include: native egg-white avidin, recombinant avidin, deglycosylated forms of avidin, bacterial streptavidin, recombinant streptavidin, truncated streptavidin, and/or any derivative thereof.

An “RNA promoter” as used herein is a DNA molecule that directs an RNA polymerase to initiate transcription.

“Ribonuclease H” is an RNase well known in the art. An RNase is an enzyme that digests RNA. RNase H is a type of RNase, and is an endoribonuclease that specifically hydrolyzes the phosphodiester bonds of RNA, when hybridized to DNA. The enzyme does not digest single stranded RNA or single stranded DNA, or double stranded DNA. In vitro uses of the enzyme is well known, in presence of suitable buffer, commercially available with the enzyme.

A “subject” generally refers to an organism that is currently living or an organism that at one time was living or an entity with a genome that can replicate. The methods, kits, and/or compositions of the disclosure can be applied to one or more single-celled or multi-cellular subjects, including but not limited to microorganisms such as bacterium and yeast; insects including but not limited to flies, beetles, and bees; plants including but not limited to corn, wheat, seaweed or algae; and animals including, but not limited to: humans; laboratory animals such as mice, rats, monkeys, and chimpanzees; domestic animals such as dogs and cats, agricultural animals such as cows, horses, pigs, sheep, goats; and wild animals such as pandas, lions, tigers, bears, leopards, elephants, zebras, giraffes, gorillas, dolphins, and whales. The methods of this disclosure can also be applied to germs or infectious agents, such as viruses or virus particles or one or more cells that have been infected by one or more viruses.

A “support” can be solid, semisolid, a bead, a surface. The support can be mobile in a solution or can be immobile.

A “transpososome” as described herein is a complex assembly comprising a transposon and other molecules helping or facilitating the transposon activity on a DNA sequence. In some embodiments, as described herein the transposon is a DNA transposon e.g. a Tn5. As described herein, for example, the transpososome introduces insert sequences within the genome of a nucleic acid, such insert sequences may comprise promoter sequences, adapter sequences, barcoding sequences, marker sequences, restriction enzyme specific sequences etc. The transpososome, comprising DNA transposon activity generates a double stranded cut at each site where it assembles. Activity of the transpososome therefore generates specific fragments from the genomic DNA. In some cases the transpososome is used to generate fragments at specific intervals in the length of the genomic DNA; additionally in some cases in a sequence specific manner, and at the same time inserting the insert sequences. In a broader context a transpososome could be an RNA transpososome, wherein the molecule is an RNA molecule, and the transposon is an RNA transposon, acting on the RNA molecule, as is known to one of skill in the art.

The term “unique identifier” may include but is not limited to a molecular bar code, or a percentage of a nucleic acid in a mix, such as dUTP.

“Repetitive sequence” as used herein refers to sequence that does not uniquely map to a single position in a nucleic acid sequence data set. Some repetitive sequence can be conceptualized as integer or fractional multiples of a repeating unit of a given size and exact or approximate sequence.

A “palindrome” or “palindromic sequence” as used herein refers to a nucleic acid sequence that is the same whether read 5′ (five-prime) to 3′ (three prime) on one strand or 5′ to 3′ on the complementary strand with which it forms a double helix.

An “inverted sequence” as used herein refers to a sequence that is the reverse sequence or reverse complement sequence relative to another sequence. A sequence is inverted if, upon (conceptually) rotating the molecule on which it is found by 180 degrees, the sequence as read in the same direction is the same sequence.

A “haplotype” as used herein refers to a collection of specific alleles in a cluster of tightly-linked genes on a chromosome that are likely to be inherited together.

A “sub-haploid” fraction as used herein refers to a genomic sample that is diluted to or that otherwise comprises less than one haploid complement of nucleic acid material.

The term “about” as used herein in reference to a number refers to a set including that number plus a range of values spanning plus or minus 10% of that number.

Before the present methods, compositions and kits are described in greater detail, it is to be understood that this invention is not limited to particular method, composition or kit described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods, compositions and kits are provided for producing multi-insert nucleic acids. These methods, compositions and kits find use in a number of application, such as whole-genome sequencing. These and other objects, advantages, and features of the invention will become apparent to those persons skilled in the art upon reading the details of the compositions and methods as more fully described below.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Preparation of Sample Nucleic Acid Into Tagmented Nucleic Acid

Methods disclosed herein comprise transposase mediated (and other types of enzyme-mediated) insertion of a nucleic acid insert sequence into a plurality of sites within individual molecules of a nucleic acid sample, thereby producing a DNA molecule comprising regions or fragments of target DNA interspersed with insertional nucleic acids. Exemplary enzymes include recombinases, such as an integrase or a transposase. In some cases the enzyme is an integrase. In some cases the enzyme is a transposase, such as Tn5 transposase. Other transposases, integrases, or recombinase enzymes are contemplated and are consistent with the disclosure herein.

Methods and compositions herein relate to multiply tagging a nucleic acid sample with an insert nucleic acid fragment used to direct PCR or other amplification of adjacent nucleic acid sequence. The sequence is used to direct RNA transcription of adjacent nucleic acid sequence into RNA that can be concurrently or subsequently reverse-transcribed into DNA that can be amplified or sequenced by downstream methods. The insertion sequence facilitates obtaining nucleic acid sequence information such as nucleic acid sequence information adjacent to the insertion site. In some alternate embodiments insert nucleic acid fragment sequence is used as a primer binding site to direct primer-extension-mediated library generation, either as an alternative or in combination.

In some embodiments, the method described herein comprise methods suitable for the generation of highly accurate linear amplification of genomic samples derived from a sample as small as a single cell or less. The amplification is highly efficient and largely uniform throughout the sample, such that as much as 90% or more of the original sample is amplified 1000× or more, and such that the vast majority of the amplified library (as much as 85% or more) is present at a level within 4× of the mean. These parameters indicate that the amplification is both very high and largely uniform throughout the sample, rendering it particularly suitable for downstream analysis.

Libraries are generated through the random introduction of promoter sequences into sample DNA. Insertion sequences are introduced through transposase treatment, recombinase treatment, invertase treatment or any other treatment, enzymatic or otherwise, that preserves phase information of the original sample. Tn5 treatment is a preferred insertion approach, but others are contemplated and consistent with the production of the libraries herein.

A benefit of the methods herein is that insertion and amplification are largely independent of the sequence of the sample prior to insertion.

Repeat regions are more easily sequenced because random insertion produces an ‘insertion fingerprint’ that renders otherwise repetitive regions unique for the purpose of library synthesis. By introducing a random insertion pattern, one is able to use the insertion pattern to map sequence reads to the locus harboring that insertion pattern. Thus repetitive regions, like loci harboring large multimeric line repeats, sine repeats, or other repetitive elements, are accurately mapped by the methods and libraries herein, while using conventional methods such loci may collapse to a single monomer at best.

Additionally, GC biases in the sample do not impact intermediate or final library synthesis, as RNA transcription is much less vulnerable to GC content than is PCR or other primer annealing based amplification approaches.

Accordingly, the methods and RNA intermediate libraries presented herein represent a substantial improvement over approaches otherwise available for the production of sequencing libraries for the sequencing of nucleic acid sample such as genomic samples.

Methods, compositions and sequence libraries are suitable for the generation of synthetic long read sequences from nucleic acid samples as small as sub-genomic samples or smaller. Starting from a small population of cells or even a single cell, nucleic acids are diluted to sub-genomic amounts prior to library generation, thereby avoiding sampling bias that may emerge when starting from bulk material, and reduce the number of compartments required on a library construction and sequencing workflow. Samples are amplified linearly, such as from nucleic acid fragments randomly inserted into the nucleic acid sample, to reduce amplification bias that may emerge from alternate amplification systems, such as pcr-based exponential sample amplification, phi-29 based amplification, or any system where a sample template is copied and the copies are used as templates for further intermediate generation. Linear amplification is accomplished through RNA polymerase promoter-directed synthesis of RNA molecules from nucleic acid fragments that have been randomly inserted into the nucleic acid sample. As the amplification intermediates are RNA molecules, they do not serve as templates for further intermediate synthesis, and in the event of hybridization to one another or to the sample, they do not prime extension to form further intermediates.

A benefit of the generation of RNA polymerase promoter-directed synthesis of RNA molecules from the nucleic acid fragments randomly inserted into the nucleic acid sample is that the resultant RNA library is derived directly from the sample nucleic acid, rather than from intermediate synthesis products that serve as templates. RNA molecules do not serve as templates for further RNA synthesis, and free RNA molecule 3′ OH moieties do not support further extension upon reannealing to the original sample, other intermediates or other regions of the molecule itself.

As a consequence, artifactual errors common to some sequencing libraries are avoided. In particular, errors in RNA molecule synthesis are not propagated, and will occur independently of one another. Thus unlike approaches involving exponential amplification of intermediates, individual errors in library synthesis are independent of one another and are each likely to be individually rare. Rare errors are easily identified in the context of the full library sequence, and are easily excluded. This is in contrast to libraries involving exponential amplification, where errors that occur in early intermediate synthesis are propagated exponentially, and can become so abundant as to be difficult to distinguish from allelic variation or rare events such as mutations in a subset of the cells of a sample, such as a tumor cell subset.

Chimeric artifacts are dramatically reduced if not effectively eliminated because the 3′ OH of synthesized RNA intermediates are not suitable for template directed extension upon their melting and reannealing in subsequent steps of synthesis. 3′ end reannealing is a substantial source of chimeric artefact formation in alternate systems, because the resulting chimeric molecules are difficult to distinguish from translocations, duplications inversions or deletions in the original sample.

Long repeat regions are resolved, even in situations where the long repeats occur at multiple loci in a sample. Insertion of nucleic acid fragments randomly inserted into the nucleic acid sample superimposes a level of uniqueness throughout the sample (an ‘insertion fingerprint’), such that sequences obtained from repetitive regions are mapped, in combination with their superimposed nucleic acid fragment random insertion patters, to unique loci of the sample. Thus, regions that are difficult to amplify, difficult to sequence and difficult to map to unique loci of a repetitive genome sample are far more easily and more accurately sequenced using the methods, compositions and libraries disclosed herein.

RNA polymerase-directed synthesis of an RNA intermediate library also avoids synthesis biases that often skew PCR-amplified libraries. RNA polymerase-directed synthesis of an RNA intermediate library is largely independent of GC concentration of the substrate, so there is a dramatic reduction in GC bias in the finalized library.

Using the methods and workflows presented herein, a sample as small as a sub-haploid genomic sample from a single cell is amplified, uniformly and to a level far above that needed for most library sequencing methods.

Alternately, some embodiments comprise annealing primers to nucleic acid fragments that have been randomly inserted into the nucleic acid sample, and extension to form library intermediates.

Provided herein are methods of sequencing a nucleic acid sample, such as a nucleic acid sample having a sequence comprising an element repeated at a first region and a second region. Some such methods comprise inserting a nucleic acid tag having a nucleic acid tag sequence into the first region at a first repeat site generating a first sequence read comprising element sequence and nucleic acid tag sequence at the first repeat site, and a second sequence read comprising element sequence spanning the first repeat site from the nucleic acid sample, and assigning the first sequence read comprising repetitive element sequence and nucleic acid tag sequence at a first repeat site to the first region. Various aspects of these methods are recited below, contemplated as distinct or in combination. Methods are contemplated to optionally include aspects wherein the repeat site comprises a position within a repetitive element or wherein the region comprises a locus of a genome that harbors a repeat site. Methods optionally comprises assigning the second sequence read comprising repetitive element sequence spanning the repeat site to the second region. It is further contemplated that the nucleic acid tag comprises RNA promoter sequence. Nucleic acid tags comprising RNA promoter sequences include at least one of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. RNA promoter sequences are contemplated to comprise a T7 sequence. Also contemplated in methods herein is inserting a nucleic acid tag having a nucleic acid tag sequence into a second site in the element at a second region. Methods may also include assigning a third sequence read comprising repetitive element sequence and comprising nucleic acid tag sequence at the second site to the second region. Methods are also contemplated comprising inserting at least two nucleic acid tags having nucleic acid tag sequences into at least two sites in the element at two or more regions at an average density of no more than 1 insertion per 500 basepairs.

Some methods contemplated herein involve converting a multimeric repeat nucleic acid region that is not uniquely sequenceable into a unique region. Some of such methods comprise treating the isolated nucleic acid sample comprising a repeated nucleic acid region that is not uniquely sequenceable a using a random insertional mutagen to insert a tag into one copy of said repeated nucleic acid region, thereby rendering said one copy of said repeated nucleic acid region unique, obtaining sequence reads from the insertionally mutagenized isolated nucleic acid sample, and assigning sequence reads having a repeated nucleic acid region sequence and a tag sequence to a unique repeated nucleic acid region. Various aspects of these methods are recited below, contemplated as distinct or in combination. Some methods optionally comprise inserting two or more nucleic acid tags having nucleic acid tag sequences into two or more sites in the element at two or more regions at an average density of no more than 1 insertion per 500 basepairs. It is further contemplated that the nucleic acid tag comprises an RNA promoter. Nucleic acid tags comprising RNA promoter sequences include at least one promoter selected from the list consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. RNA promoter sequences are contemplated to comprise at least one of T7, T3 and SP6. RNA promoter sequences are also contemplated to comprise T7. It is further contemplated that the method comprises contacting said insertionally mutagenized isolated nucleic acid sample to an RNA polymerase. Also contemplated in methods herein is generation of a population of RNA molecules comprising tag sequence and repeated nucleic acid region sequence. Methods also include sequence reads, wherein the sequence reads are obtained from the population of RNA molecules. Further contemplated are methods wherein the population of RNA molecules is reverse transcribed to generate DNA molecules. Methods herein are also contemplated to comprise aspects wherein the random insertional mutagen comprises a transposase. Contemplated herein are methods wherein the transposase is at least one transposase selected from the list consisting of Tn5 transposase, sleeping beauty transposase, piggybac transposase, and Mariner transposase. Transposases contemplated herein comprise a Tn5.

Also provided herein are isolated nucleic acid samples, such as isolated nucleic acid samples treated with an insertional mutagen. Some such nucleic acid samples comprise a first repeat element interrupted by a tag at a first position and a second copy of said repeat element is interrupted by said tag at a second position, such that sequence reads comprising tag sequence and repeat element sequence indicative of said tag at said first position uniquely map to said first repeat element. Various aspects of these nucleic acid samples are recited below, contemplated as distinct or in combination. Nucleic acid samples contemplated herein optionally comprise two or more nucleic acid tags having nucleic acid tag sequences at two or more sites in the repeat element at two or more regions at an average density of no more than 1 insertion per 500 basepairs. Nucleic acid tags comprising RNA promoter sequences include at least one of the list consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. RNA promoter sequences are contemplated to comprise at least one of the list consisting of a T7, T3, and SP6. RNA promoter sequences are contemplated to comprise a T7 promoter. Insertional mutagens of the nucleic acid samples contemplated herein a transposase selected from the list consisting of Tn5 transposase, sleeping beauty transposase, piggybac transposase, and Mariner transposase. Alternatively, insertional mutagens contemplated herein comprise an integrase. Repeat elements of the nucleic acid samples contemplated herein are selected from the group consisting of a transposon, a retrotransposon, a DNA transposon, an insertion sequence, a plasmid, a bacteriophage, a group II intron, a group I intron, an Alu element, a MIR element, an intracisternal A particle (IAP), an ETn, a virus, a transposable element, a LINE, and a SINE. Nucleic acid samples herein are contemplated to optionally be used to create an RNA library generated by contacting a nucleic acid sample to an RNA polymerase. Alternatively, nucleic acid samples herein are contemplated to be used to create a DNA library generated by contacting a RNA library herein to a reverse-transcriptase.

Additional nucleic acid samples contemplated herein include a genomic nucleic acid sample sequencing library comprising a plurality of RNA molecules. Some such plurality of RNA molecules comprise a first end comprising tag sequence and a second end comprising genomic nucleic acid sample sequence, wherein at least 90% of said genomic nucleic acid sample such as a human genomic sample is represented in said plurality of RNA molecules. Various aspects of these nucleic acid sequencing libraries are recited below, contemplated as distinct or in combination. Also provided, nucleic acid sequencing libraries contemplated herein comprise a plurality of RNA molecules generated directly from the genomic nucleic acid sample. Optionally, such nucleic acid sequencing libraries comprise at least 95% of said genomic nucleic acid sample is represented in said plurality of RNA molecules. Alternatively, such nucleic acid sequencing libraries comprise at least 99% of said genomic nucleic acid sample is represented in said plurality of RNA molecules. Nucleic acid sequencing libraries provided herein also comprise a sample wherein said sample is amplified at least 100× relative to said genomic sample. Alternatively, nucleic acid sequencing libraries provided herein comprise a sample wherein said sample is amplified at least 1000× relative to said genomic sample. Also provided in nucleic acid sequencing libraries herein are sequencing libraries wherein at least 85% of said amplified sample is present at a level that is no more than 4× of a mean amplification level. Nucleic acid sequencing libraries contemplated herein include libraries wherein said RNA promoter sequence comprises at least an identifiable portion promoter is at least one promoter selected from the list consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. RNA promoter sequences in nucleic acid sequencing libraries herein alternatively comprise at least one of T7, T3 and SP6. RNA promoter sequences in nucleic acid sequencing libraries optionally comprise a T7 promoter sequence. Also provided herein are nucleic acid sequencing libraries wherein said genomic nucleic acid sample is treated to insert a nucleic acid encoding said RNA promoter sequence into said genomic nucleic acid sample. Optionally, nucleic acid libraries include libraries comprising a genomic nucleic acid sample contacted to an integrase. Alternatively, nucleic acid libraries include libraries comprising a genomic nucleic acid sample contacted to a transposase. Transposases contacted to a genomic nucleic acid sample in nucleic acid sequencing libraries contemplated herein comprise a transposase is selected from the list consisting of Tn5 transposase, sleeping beauty transposase, piggybac transposase, and Mariner transposase. Optionally, transposases contacted to genomic nucleic acid samples in nucleic acid sequencing libraries include Tn5. Included in nucleic acid sequencing libraries, are DNA libraries, such as a DNA library comprising a RNA library provided herein contacted to a reverse-transcriptase.

Nucleic acid samples herein are also contemplated to include genomic nucleic acid sample sequencing libraries, such as a genomic nucleic acid sample sequencing library comprising a plurality of RNA molecules. Such plurality of RNA molecules are transcribed directly from the genomic nucleic acid sample, such that no RNA molecule serves as a template for a second RNA molecule. Various aspects of these nucleic acid sequencing libraries are recited below, contemplated as distinct or in combination. For example, genomic nucleic acid sample sequencing libraries are contemplated to comprise populations wherein at least 90% of said genomic nucleic acid sample is represented in said library. Some genomic nucleic acid sample sequencing libraries are contemplated to comprise at least 95% of said genomic nucleic acid sample is represented in said library. Optionally, genomic nucleic acid sequencing libraries comprise at least 99% of said genomic nucleic acid sample is represented in said library. It is further contemplated that genomic nucleic acid sample sequencing libraries comprise a sample is amplified at least 100× relative to said genomic sample. Alternatively, it is contemplated that said sample is amplified at least 1000× relative to said genomic sample. Optionally, the genomic nucleic acid sample sequencing library comprises a library wherein at least 85% of said amplified sample is present at a level that is no more than 4× of a mean amplification level. Genomic nucleic acid sample sequencing libraries herein further comprise at least one promoter selected from the list consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. Alternatively, genomic nucleic acid sequencing libraries include an RNA promoter sequence comprising at least one of T7, T3 and SP6. Optionally, genomic nucleic acid sequencing libraries include an RNA promoter sequence comprising a T7 promoter sequence.

Also provided herein are isolated nucleic acid samples, such as nucleic acid samples comprising an isolated genomic nucleic acid sample into which a exogenous promoter is inserted at an average density of at least 1 insertion per 5 kb. Various aspects of these isolated nucleic acid samples are recited below, contemplated as distinct or in combination. For example, nucleic acid samples contemplated herein include samples wherein the exogenous promoter is inserted at an average density of no more than 1 insertion per 500 basepairs. Optionally, the nucleic acid sample is contacted to an RNA polymerase. Nucleic acid samples herein are also contemplated to include samples comprising a plurality of RNA molecules comprising exogenous promoter sequence and isolated genomic nucleic acid sample sequence. Optionally, nucleic acid samples are contemplated to include samples wherein 90% of the isolated genomic nucleic acid sample sequence is represented by said plurality of RNA molecules. Alternatively, 95% of the isolated genomic nucleic acid sample sequence is represented by said plurality of RNA molecules. Alternatively, 99% of the isolated genomic nucleic acid sample sequence is represented by said plurality of RNA molecules. Also contemplated herein, nucleic acid samples are amplified at least 100× relative to said genomic sample. Optionally, nucleic acid samples are amplified at least 1000× relative to said genomic sample. Further nucleic acid samples contemplated comprise at least 85% of said amplified sample is present at a level that is no more than 4× of a mean amplification level.

Also provided herein are isolated nucleic acid samples, such as a nucleic acid sample comprising a plurality of repetitive elements having a length of at least 300 to 500 base pairs, wherein at least 50%, 60%, 70%, 80%, or 90% or greater than 90% of said plurality of repetitive elements are independently interrupted by at least one species of randomly inserted tag. Various aspects of these isolated nucleic acid samples are recited below, contemplated as distinct or in combination. For example, nucleic acid samples wherein the plurality of repetitive elements have a length of at least 6000 base pairs. Nucleic acid samples are also contemplated to include at least one species of randomly inserted tag comprising a nucleic acid encoding a promoter sequence. RNA promoters included in nucleic acid samples herein optionally include at least one promoter selected from the list consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. RNA promoters herein optionally comprise at least one of T7, T3 and SP6. RNA promoters alternatively comprise a T7 promoter sequence. Nucleic acid samples herein are also contemplated to include samples wherein said sample is contact to an RNA polymerase. Further contemplated of nucleic acid samples herein are samples wherein RNA molecules representing at least 90% of said nucleic acid sample are generated. Alternatively, RNA molecules representing at least 95% of said nucleic acid sample are generated. Optionally, RNA molecules representing at least 99% of said nucleic acid sample are generated. Also contemplated is a nucleic acid sample wherein said sample is subsequently contacted to a DNase.

Methods provided herein also include methods of generating a modified nucleic acid. Some such methods comprise combining an insertional nucleic acid comprising an adapter sequence that is flanked by nucleic acid integrase recognition sequences; a target nucleic acid molecule; and a nucleic acid integrase, wherein the nucleic acid integrase covalently inserts the insertional nucleic acid into the target nucleic acid at a first location and at a second location within the target nucleic acid molecule, said first location and said second location being separated by at least 200 bp. Various aspects of these methods are recited below, contemplated as distinct or in combination. Methods optionally comprise said first location and said second location are separated by at least 500 bp. Alternatively, said first location and said second location are separated by at least 750 bp, 1.0 kb, 1.5 kb, 2.0 kb, 2.5 kb. Alternatively, said first location and said second location are separated by at most 2.0 kb, 1.5 kb, or 1 kb or less than 1 kb.

Methods provided herein also include methods of generating a plurality of multi-insert nucleic acids. Some such methods comprise combining an insertional nucleic acid comprising an adapter sequence that is flanked by nucleic acid integrase recognition sequences a plurality of nucleic acids comprising a sequence of interest, e.g., a target sequence within a sample; and a nucleic acid integrase, wherein the nucleic acid integrase cleaves one or more of the plurality of fragmented nucleic acids to produce one or more recombination sites, recognizes the nucleic acid integrase recognition sequences; and inserts the insertional nucleic acid into the one or more recombination sites to generate the plurality of multi-insert nucleic acids. Various aspects of these methods are recited below, contemplated as distinct or in combination. Contemplated herein are methods wherein the adapter sequence comprises an RNA promoter sequence. Further contemplated are methods wherein the adapter sequence comprises at least one of T7, T3 and SP6 RNA promoter sequence. Also contemplated are methods wherein the adapter sequence comprises T7 RNA promoter sequence. Methods contemplated herein optionally further comprise adding a PCR primer to the plurality multi-insert nucleic acids, wherein the PCR primer anneals to the insertional nucleic acid or a portion thereof, and amplifying one or more of the plurality of multi-insert nucleic acids or a portion thereof. Optionally, the PCR primer anneals to the adapter sequence or portion thereof. Methods contemplated herein alternatively further comprise diluting the plurality of multi-insert nucleic acids into a plurality of containers, to produce a first plurality of diluted multi-insert nucleic acids in a first container and a second plurality of diluted multi-insert nucleic acids in a second container. Methods contemplated herein optionally comprise diluting the plurality of multi-insert nucleic acids into a plurality of containers dilutes the plurality of multi-insert nucleic acids such that a single multi-insert nucleic acid is present in each container of the plurality of containers. In methods contemplated herein the plurality of multi-insert nucleic acids optionally comprises genomic DNA, and wherein diluting the plurality of multi-insert nucleic acids into a plurality of containers dilutes the genomic DNA such that a haplotype frequency in a container is very low. Alternatively, in methods herein the plurality of containers comprises a container selected from a tube, a microwell and a droplet. Optionally, methods of generating a plurality of multi-insert nucleic acids further comprise providing a first PCR primer comprising a first tag to the first container, wherein at least a portion of the first PCR primer anneals the insertional nucleic acid or portion thereof; providing a second PCR primer comprising a second tag to the second container, wherein at least a portion of the second PCR primer anneals to the insertional nucleic acid or portion thereof, and wherein the second tag is different than the first tag; providing a nucleic acid polymerase into the first container and the second container; amplifying the first plurality of diluted multi-insert nucleic acids or portions thereof, thereby producing a first plurality of tagged nucleic acids; and amplifying the second plurality of diluted multi-insert nucleic acids or portions thereby producing a second plurality of tagged nucleic acids. Alternatively, in such methods herein the first tag comprises a first tag nucleic acid sequence and the second tag comprises a second tag nucleic acid sequence, wherein the first tag nucleic acid sequence and the second tag nucleic acid sequence are different.

In some embodiments the tag nucleic acid sequences comprise a promoter sequences such as a T7 promoter.

Optionally, the nucleic acid polymerase is a phi29 DNA polymerase and the insertional nucleic acid comprises random primer annealing sites.

In some embodiments, the nucleic acid polymerase is T7 polymerase and the insertional nucleic acid comprises a T7 primer annealing site.

In some embodiments, one or more synthetic adapter sequences comprising signature sequences or barcodes or UMI codes are inserted downstream of the promoter sequences.

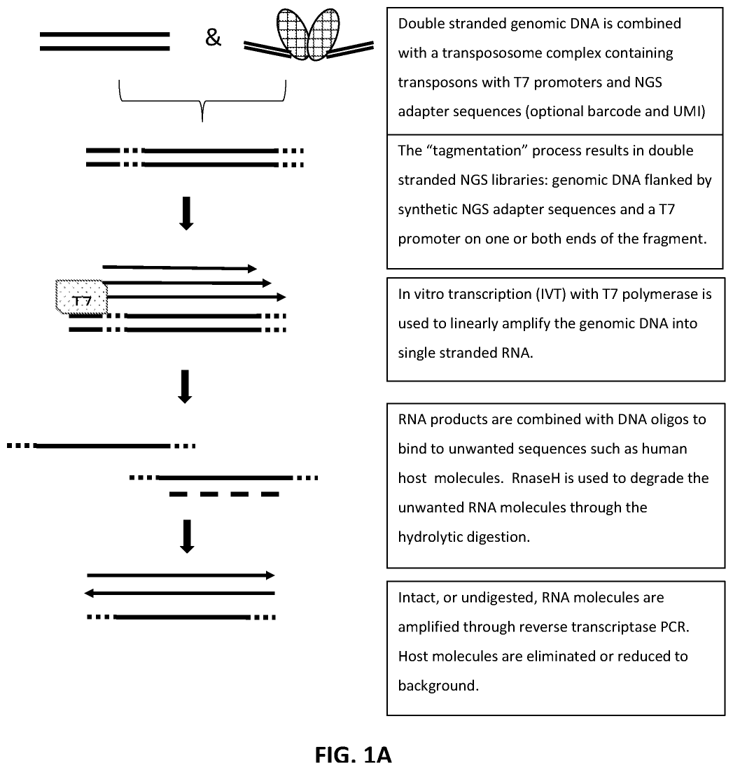

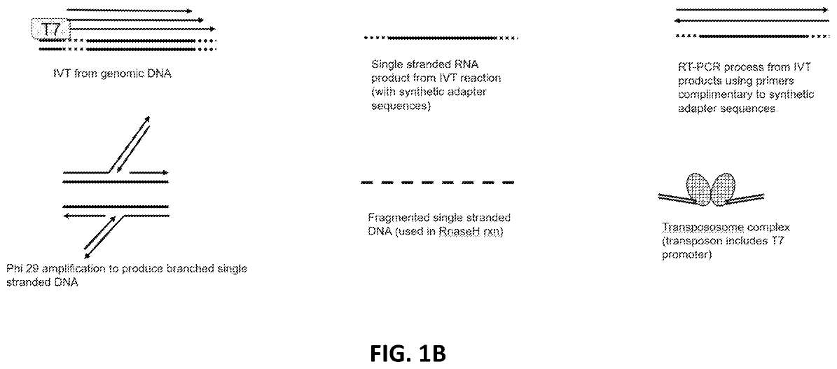

In some embodiments, the transposase inserts a sequence comprising a promoter and at least one synthetic adapter or primer sequence in the DNA at each of the transpososome complex insertional site (FIGS. 1A and 1B).

Methods contemplated herein optionally further comprise introducing a reverse transcriptase and random primers. Optionally, methods contemplated herein further comprise cleaving the insertional nucleic acid of the plurality of multi-insert nucleic acids to produce a plurality of multi-insert nucleic acid fragments, wherein each multi-insert nucleic acid fragment is flanked by the first portion of the insertional nucleic acid and the second portion of the insertional nucleic acid. Optionally, methods are contemplated wherein the cleaving occurs before adding the first and/or second PCR primer and the amplifying. Alternatively, methods herein further comprise pooling the first plurality of tagged nucleic acids and the second plurality of tagged nucleic acids. Methods herein optionally further comprise adding an affinity molecule to the first plurality of tagged nucleic acids and/or the second plurality of tagged nucleic acids. Optionally, the affinity molecule is biotin. Also contemplated herein are methods further comprising capturing the first plurality of tagged nucleic acids and/or the second plurality of tagged nucleic acids via the affinity molecule. Alternatively, methods herein further comprise sequencing the first plurality of tagged nucleic acids and the second plurality of tagged nucleic acids. Also contemplated herein are methods wherein the first portion of the insertional nucleic acid comprises a first portion of the adapter sequence and the second portion of the insertional nucleic acid comprises a second portion of the adapter sequence. Optionally, the first portion of the adapter sequence and the second portion of the adapter sequence comprise a different sequence. Alternatively, the first portion of the adapter sequence is the same as the second portion of the adapter sequence. Alternatively, the first portion of the adapter sequence and the second portion of the adapter sequence are adjacent prior to combining the insertional nucleic acid, plurality of nucleic acids and integrase. Optionally, the first portion of the adapter sequence is an inverted sequence of the second portion of the adapter sequence. Alternatively, the first portion of the adapter sequence and the second portion of the adapter sequence form a palindromic sequence. Optionally, the nucleic acid integrase is a transposase. Optionally, herein, the transposase is a Tn5 transposase. Alternatively, herein, the nucleic acid integrase recognition sequences are mosaic ends. Methods contemplated herein alternatively comprise the ratio of transposase to insertional nucleic acid set such that insertional nucleic acids are introduced at an average density of 500 bp to 2 kb over a span of at least 3 insertional nucleic acid insertion sites.

Also provided herein are nucleic acid molecules, such as nucleic acid molecules comprising a chromosome-sized target nucleic acid and a plurality of insertional nucleic acids, wherein the plurality of insertional nucleic acids are distributed at a plurality of recombination sites throughout the target nucleic acid at an average density of at least one insert per 10 kb. Various aspects of these nucleic acid molecules are recited below, contemplated as distinct or in combination. Nucleic acid molecules contemplated herein comprise molecules wherein the insertional nucleic acid comprises a primer annealing sequence. Optionally, the insertional nucleic acid comprises a first primer annealing sequence and a second primer annealing sequence. Alternatively, the first primer annealing sequence and the second primer annealing sequence are adjacent. Alternatively, the first primer annealing sequence and the second primer annealing sequence are different. Alternatively, the first primer annealing sequence is an inverted sequence of the second primer annealing sequence. Alternatively, the first primer annealing sequence and the second primer annealing sequence comprise the same sequence. Alternatively, the first primer annealing sequence and the second primer annealing sequence form a palindrome. Optionally, the insertional nucleic acid comprises a transcriptional promoter. Nucleic acid molecules herein are also contemplated wherein the insertional nucleic acid encodes a promoter selected from the list of promoters consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6. Optionally, the insertional nucleic acid encodes a promoter selected from the list of promoters consisting of a T7, T3, and SP6. Optionally, the insertional nucleic acid encodes a T7 promoter. Nucleic acid molecules herein are contemplated wherein the transcriptional promoter is recognized by an RNA polymerase. Optionally, the insertional nucleic acid comprises a mosaic end, wherein the mosaic end is recognized by a transposase. Nucleic acid molecules are also contemplated herein wherein the sample nucleic acid or nucleic acid of interest comprises a plurality of nucleic acid fragments separated by one or more of the insertional nucleic acids of the plurality of inserted nucleic acids. Optionally, nucleic acid molecules are contemplated wherein each insertional nucleic acid of the plurality of insertional nucleic acids occurs at an average frequency of about 500 base pairs to about 2000 base pairs within the target nucleic acid. Optionally, the target nucleic acid comprises DNA. Optionally, the sample nucleic acid is DNA. Optionally the sample nucleic acid comprises genomic DNA. Optionally, the DNA is mammalian DNA. Optionally the sample nucleic acid is cDNA. Optionally, the sample nucleic acid is an RNA. Optionally the sample nucleic acid is total RNA. Optionally, the sample nucleic acid mRNA. The sample nucleic acid, when RNA the first conversion step to RNA is unnecessary as is known to one of skill in the art. A cleaning up of the sample may be necessary prior to performing any subsequent steps

Also provided herein are insertional nucleic acid molecules, such as insertional nucleic acids comprising an adapter sequence and two mosaic ends, wherein the mosaic ends are recognized by a transposase. Various aspects of these nucleic acid molecules are recited below, contemplated as distinct or in combination. Optionally, contemplated herein are insertional nucleic acids wherein the adapter sequence comprises a first primer binding site and a second primer binding site, wherein the first primer binding site and a second primer binding site are adjacent, and the two mosaic ends flank the adapter sequence. Alternatively, the first primer binding site is an inverted sequence of the second primer binding site. Alternatively, the first primer binding site is a palindromic sequence of the second primer binding site. Alternatively, the first primer binding site and the second primer binding site comprise a different sequence. Also contemplated herein are insertional nucleic acids wherein the insertional nucleic acid, from 5′ to 3′, comprises a first mosaic end, a first primer binding site, a second primer binding site and a second mosaic end. Optionally, the adapter sequence comprises a transcriptional promoter.

Also provided herein are kits comprising insertional nucleic acids, such as kits comprising an insertional nucleic acid, wherein the oligonucleotide comprises a mosaic end that is recognized by a transposase; and a transposase. Various aspects of these kits are recited below, contemplated as distinct or in combination. Optionally contemplated herein the insertional nucleic acid further comprises an adapter sequence. Alternatively, the adapter sequence is flanked by a first mosaic end and a second mosaic end. Alternatively, the adapter sequence comprises a primer annealing sequence. Alternatively, kits contemplated herein further comprise a PCR primer that anneals to the primer annealing sequence. Optionally, the PCR primer comprises a tag. Alternatively contemplated herein are kits wherein a first PCR primer comprises a first tag and a second PCR primer comprises a second tag, wherein the first tag and the second tag are different. Optionally contemplated herein are kits further comprising a plurality of containers. Alternatively, the plurality of containers comprises a microwell plate. Also contemplated herein are kits wherein one or more containers of the plurality of containers contains a mixture comprising one or more of the transposase, a portion of the plurality of insertional nucleic acids and the first/second PCR primers. Alternatively, the transposase is a Tn5 transposase. Optionally contemplated herein are kits further comprising a polymerase.

Also provided herein are nucleic acid molecules, such as nucleic acid sequence of interest that are isolated, segregated or removed from unwanted nucleic acid in the sample, the method comprising at step in which nucleic acid molecules comprising a first nucleic acid insert sequence is inserted at a first insertion site, a first nucleic acid insert sequence inserted at a second insertion site, and a first nucleic acid sequence at a third insertion site, wherein said first insertion site and said second insertion site are separated by at least 250 bp of nucleic acid molecule sequence that is not first nucleic acid insert sequence. Various aspects of preparing these nucleic acid molecules are recited below, contemplated as distinct or in combination. Optionally contemplated herein said first nucleic acid insert sequence comprises a left border and a right border bound by a transposase. Alternatively contemplated herein said left border is bound by a transposase if not covalently linked to flaking sequence on either side of said left border. Alternatively, said right border is bound by a transposase if not covalently linked to flaking sequence on either side of said right border. Optionally, target nucleic acids are contemplated wherein a transposon directs covalent insertion of a molecule having said first nucleic acid insertion sequence into a nucleic acid molecule to generate a nucleic acid molecule comprising nucleic acid from the sample that comprises e.g., a sequence of interest, in combination with unwanted sequences. Additionally, and alternatively, a second insertion site and a third insertion site are separated by at least 250 bp of nucleic acid molecule sequence that is not first nucleic acid insert sequence. Optionally contemplated herein are nucleic acid molecules comprising a fourth insertion site, wherein said third insertion site and said fourth insertion site are separated by at least 250 bp of nucleic acid molecule sequence that is not first nucleic acid insert sequence. Alternatively, a first insertion site and a second insertion site are separated by at most 2.5 kb. Alternatively, said first nucleic acid insert sequence comprises a first primer binding site. Also contemplated herein are nucleic acid molecules, wherein said first nucleic acid insert sequence comprises a palindromic sequence such that a first primer binding site is present in a first orientation and a second orientation, said second orientation being antipolar to said first orientation. Optionally, said first nucleic acid insert sequence comprises a restriction endonuclease cleavage site between said first primer binding site orientation and said second primer binding site orientation. Alternatively, said first nucleic acid insert sequence comprises a first primer binding site and a second primer binding site. Alternatively, said first nucleic acid insert sequence comprises a restriction endonuclease cleavage site between said first primer binding site and said second primer binding site. Alternatively, said first nucleic acid insert sequence comprises an RNA polymerase promoter. Optionally, the RNA polymerase promoter is a T7 RNA polymerase promoter.

Also provided herein are compositions, such as compositions comprising a nucleic acid molecule comprising a first nucleic acid insert sequence at a first insertion site, a first nucleic acid insert sequence at a second insertion site, and a first nucleic acid sequence at a third insertion site, wherein said first insertion site and said second insertion site are separated by at least 250 bp of nucleic acid molecule sequence that is not first nucleic acid insert sequence, and a population of oligonucleotide primers, said population of oligonucleotide primers comprising a plurality of oligonucleotide primers each having sequence reverse complementary to said first nucleic acid insert sequence, and each of said plurality of oligonucleotide primers having a common barcode sequence. Various aspects of these compositions are recited below, contemplated as distinct or in combination. Compositions herein optionally are contemplated wherein said common barcode sequence corresponds to said nucleic acid molecule. Alternatively, said common barcode sequence corresponds to a container of said composition. Alternatively, said common barcode sequence corresponds to at least one container of a plurality of containers of said composition. Compositions herein are optionally contemplated wherein said container is a well in a multiwell plate. Alternatively, said container is a droplet. Alternatively, said container is a micelle.

Methods provided herein also include methods of assigning nucleic acid molecule-specific sequence information. Some such methods comprise: obtaining a nucleic acid sample comprising a nucleic acid molecule, inserting an insertion sequence into said nucleic acid molecule at a first site, amplifying nucleic acid molecule sequence adjacent to said first site, and sequencing said nucleic acid molecule sequence adjacent to said first site. Various aspects of these methods are recited below, contemplated as distinct or in combination. Contemplated herein are methods wherein optionally inserting said insertion sequence comprises contacting said nucleic acid with a nucleic acid integrase. Alternatively, said nucleic acid integrase comprises a transposase. Methods contemplated herein optionally comprise inserting an insertion sequence into said nucleic acid molecule at a second site, said second site separated from said first site by about 500 bp to 3 kb. Alternatively, said amplifying comprises contacting said insertion sequence with a first primer that anneals to said first insertion sequence at said first insertion site. Alternatively, said amplifying comprises contacting said insertion sequence with a second primer that anneals to said first insertion sequence at a second insertion site. Alternatively, methods herein comprise segregating said nucleic acid sample among a plurality of partitions prior to said amplifying. Optionally, said first primer sequence comprises a first tag that corresponds to a subset of said plurality of said partitions. Alternatively, said second primer sequence comprises a second tag that corresponds to a subset of said plurality of said partitions. Alternatively, said first tag and said second tag comprise identical sequence. Alternatively, said first tag and said second tag comprise non-identical sequence. Methods herein alternatively comprise contacting said first insertion sequence to an RNA polymerase prior to said amplifying. Optionally, said RNA polymerase is a T7 RNA polymerase. Methods herein optionally comprise contacting said first insertion sequence with DNase subsequent to contacting to an RNA polymerase. Methods herein optionally comprise contacting said first insertion sequence with reverse-transcriptase subsequent to contacting to an RNA polymerase. Alternatively, contacting said first insertion sequence with reverse-transcriptase concurrently with contacting said first insertion sequence with RNA polymerase.

### A. Introducing Insertional Nucleic Acids Into Sample Nucleic Acids to Produce Multi-Insert Nucleic Acids

Disclosed herein are methods of generating a plurality of multi-insert nucleic acids, the methods comprising combining: an insertional nucleic acid that comprises an RNA polymerase promoter flanked by nucleic acid integrase recognition sequences; a plurality of nucleic acids comprising a target nucleic combined with contaminating or unwanted (non-target nucleic acid); and a nucleic acid recombinase such as a transposase, wherein the nucleic acid integrase cleaves one or more of the plurality of nucleic acids to produce one or more recombination sites within the target nucleic acids; recognizes the nucleic acid integrase recognition sequences; and inserts the insertional nucleic acid into the one or more insertion sites to generate the plurality of multi-insert nucleic acids. Conditions for transposase or other recombinase or invertase activity are known to those of skill in the art.

Insertion reactions are performed using reagents at concentrations so as to effect a desired insertion density in the sample. Desired insertion densities vary, but are often in the range of one insert for each 500 bp to 2 kb, 3 kb, 4 kb, or 5 kb or greater. Desired insertion density often varies with RNA synthesis extension success, such that conditions resulting in longer RNA intermediate molecules, such as 10 kb, 20 kb, 30 kb or greater, facilitate less dense insertion events. In alternate cases, the one or more insertion sites are separated by an average distance selected from less than 100 bp, less than 200 bp, less than 300 bp, less than 400 bp, less than 500 bp, less than 600 bp, less than 700 bp, less than 800 bp, less than 900 bp, less than 1000 bp, less than 1100 bp, less than 1200 bp, less than 1300 bp, less than 1400 bp, less than 1500 bp, less than 1600 bp, less than 1700 bp, less than 1800 bp, less than 1900 bp, less than 2000 bp, less than 2100 bp, less than 2200 bp, less than 2300 bp, less than 2400 bp, less than 2500 bp, less than 2600 bp, less than 2700 bp, less than 2800 bp, less than 2900 bp and less than 3000 bp. In some cases, insertion sites are separated by an average distance of about 500 bp. In some cases, insertion sites are separated by an average distance of about 1000 bp. In some cases, insertion sites are separated by an average distance of about 1500 bp. In some cases, insertion sites are separated by a distance dependent on the ratio of target nucleic acid to integrase.

### Nucleic Acids, Sample and Target Sequence

The methods disclosed herein comprise insertional modification of one or more sample nucleic acids comprising a target nucleic acid. Exemplary target nucleic acid comprises DNA, such as double stranded DNA. Target nucleic acid(s) often comprise genomic DNA, or cDNA libraries or other DNA sources. A wide range of organisms are suitable sources for genomic DNA, such as eukaryotic, eubacterial or archaeal sources. Non-limiting sample sources include animal DNA, plant DNA, or generally, DNA is isolated from a biological sample such as a blood sample, a bodily fluid sample, a hair sample, a skin sample, a saliva sample, etc., as indicated elsewhere herein and as contemplated by one of skill in the art.

A sample comprising a target nucleic acids can be obtained from a population of cells. For example some target nucleic acids are obtained from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 cells, or more than 100 cells. The methods and compositions herein are consistent with obtaining target nucleic acids from a single cell, and even subdividing the target nucleic acids into sub genomic fractions.

Target nucleic acids are provided at an amount selected from about 1 pg, 2 pg, 3 pg, 3.2 pg, 4 pg, 5 pg, 6 pg, 7 pg, 8 pg, pg, 10 pg, 20 pg, 30 pg, 40 pg, 50 pg, 60 pg, 70 pg, 80 pg 90 pg, 100 pg, 200 pg, 300 pg, 400 pg, 500 pg, 600 pg, 700 pg, 800 pg, 900 pg, 1 ng, 2 ng, 3 ng, 4 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 21 ng, 22 ng, 23 ng, 24 ng, 25 ng, 26 ng, 27 ng, 28 ng, 29 ng, 30 ng, 31 ng, 32 ng, 33 ng, 34 ng, 35 ng, 36 ng, 37 ng, 38 ng, 39 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 61 ng, 62 ng, 63 ng, 64 ng, 65 ng, 66 ng, 67 ng, 68 ng, 69 ng, 70 ng, 71 ng, 72 ng, 73 ng, 74 ng, 75 ng, 76 ng, 77 ng, 78 ng, 79 ng, 80 ng, 81 ng, 82 ng, 83 ng, 84 ng, 85 ng, 86 ng, 87 ng, 88 ng, 89 ng, 90 ng, 91 ng, 92 ng, 93 ng, 94 ng, 95 ng, 96 ng, 97 ng, 98 ng, 99 ng or 100 ng, and a value outside of the range defined by the above-mentioned list. Often, the target nucleic acids are obtained/provided at an amount of about 50 ng.

### Insertional Nucleic Acids

Disclosed herein are methods of inserting an insertional nucleic acid into a sample nucleic acid comprising target nucleic acid. Some methods comprise introducing a plurality of insertional nucleic acids into a sample nucleic acid or a portion thereof. Often, each insertional nucleic acid of the plurality of insertional nucleic acids consists of a same sequence. Alternately, one or more of the insertional nucleic acids of the plurality of insertional nucleic acids consist of a different sequence.

Some insertional nucleic acids disclosed herein have a minimal nucleotide length while having the ability to insert into a target nucleic acid. For example, core insertional nucleic acids have a left border nucleic acid, an RNA promoter such as a T7, T3, or SP6 promoter and a right border nucleic acid. Some insertional nucleic acid described herein are transposable nucleic acid elements, such as a Tn5 transposon comprising the necessary left and right mosaic end sequences necessary for transposition and an RNA promoter, such as a T7, T3, or SP6 promoter. Insertional nucleic acids optionally comprise a tag sequence. In some cases, the insertional nucleic acid comprises two RNA promoters, such as two T7, T3, or SP6 promoters which direct transcription in opposite directions. In some cases, the insertional nucleic acid comprises two different RNA promoters, such as a T7 and a T3, a T7 and a SP6, or a T3 and a SP6 promoter which direct transcription in opposite directions. Such insertional nucleic acids allow from RNA transcription to occur in two directions from one location in the target nucleic acid. This is particularly useful when sequencing certain types of target nucleic acid such as repeat regions and telomeres.

A number of insertion nucleic acid fragments are consistent with the disclosure herein. A core nucleic acid fragment comprises a left and right border required for transposase binding, transpososome assembly, and insertion into a sample such as a genomic nucleic acid sample, and also comprises an RNA polymerase promoter sequence. Thus, in preferred minimal insertion fragment examples, an insert comprises 60 or fewer base pairs, comprising a first transposase border of as few as 23, 22, 21, 20, 19, 18, 17 bases or fewer, an RNA promoter such as a T7 promoter of 25, 24, 23, 22, 21, 20, 19, 18, 17 or fewer bases, and a second transposase border, for a total in some cases of fewer than 60 bases. Small inserts are preferred because they occupy a smaller proportion of the total sequence reads of the sample when formatted into a sequencing library and sequenced, although in alternate cases a larger insert comprising additional nucleic acid sequence or information is employed

The insertional nucleic acid ends comprise mosaic ends (ME) or other sequence ends that are recognized by the transposase or other insertional enzymes and required for insertion.

Nested between mosaic ends are found one or more RNA polymerase promoters or other sequence. As discussed herein, T7, T3, and SP6 are preferred in many cases, but a broad range of RNA promoters are contemplated, including T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, U6, and other RNA promoters known in the art.

In minimal insert examples, the insert comprises a single RNA promoter bounded by an ME or other transposase or invertase or recombinase sequence at either end. In these examples insert size is minimized, for example to minimize the amount of insertion sequence in the final sequenced library. Alternately, insertions further include a second RNA polymerase promoter site, such as a site positioned antiparallel to the first so that they direct RNA polymerase extension in opposite directions from the insertion. Such a configuration effectively doubles the number of RNA intermediates generated from a single whole-sample insertion event. Insertions optionally include a barcode sequence, optionally positioned so as to be transcribed, so that the transcription fragments are differentially barcoded by insertion sequence. In alternate embodiments or in combination, library constituents are barcoded through downstream processing of the library RNA intermediates such as during their reverse-transcription into a DNA library.

Some alternative insertion strategies involve insertion populations, whereby the majority of inserts are as described above, but are provided with a percentage of ‘gapped’ insert whereby the left and right borders are not connected in a loaded transposon. These gapped inserts are effective if some sample fragmentation is desired, as their insertion results in breakage and local loss of phase information. In some cases gapped inserts are employed at a frequency of 1%, 2%, 3%, 4%, 5%, 6%, 7%, 8%, 9%, 10% or other proportion of the total insert population.

Some alternative insertion strategies involve inserts having transcription termination sites upstream of the promoter sequence, so as to prevent read-through from transcripts extending from upstream of the promoter. Such inserts are preferred if one wants to limit overall or average transcript extension. Much like the ‘gapped’ inserts, above, inserts having transcription termination sites are often used in insertion populations, for example at a frequency of 1%, 2%, 3%, 4%, 5%, 6%, 7%, 8%, 9%, 10% or other proportion of the total insert population

Some alternate embodiments rely upon primer annealing and extension rather than RNA polymerase-driven RNA synthesis alone. These embodiments comprise one or more of the following elements, alone or in combination. The double stranded insertional nucleic acid comprises an adapter sequence between the mosaic ends. The adapter sequence comprises a first adapter sequence and a second adapter sequence. Thus, the methods result in producing multiple contiguous DNA sequences, where the inverted adapter sequences are split by stretches of gDNA of a desired length (e.g. 5′-19 bp ME←A adapter-B adapter→-gDNA insert-19 bp ME-←A adapter-B adapter→ . . . repeat N times through full length contiguous sequence). The primer binding site and the ME sequence may overlap. Some insertion sequences additionally have a tag sequence adjacent to the ME sequence such that amplification from the primer results in an amplicon having a barcode sequence. The barcode sequence is unique to the insert, or alternatively is common to all inserts introduced at a given iteration of an iterative insertion process. The first adapter sequence and the second adapter sequence are arranged in an inverted configuration (e.g. inverted adapter sequences) or a palindromic configuration. The adapter sequences may comprise a primer annealing sequence. Primers that anneal to the adapter sequences are designed to amplify the genomic DNA between the insertional sequences, as indicated by the arrows in the example above. Some primers comprise a tag. The tag is optionally specific to a subset of the genomic DNA. Some methods comprise sequencing the amplified genomic DNA. Sequencing may comprise a next generation sequencing (NGS) method or modification thereof.

Some insertion nucleic acid fragments are synthesized to include a preferred restriction endonuclease or other cleavage site, so that nucleic acid samples treated with an insert can be cleaved at insertion sites.

Thus, both preferred RNA polymerase promoter-directed library generation and primer-extension mediated library generation are contemplated herein. These approaches are not mutually inconsistent, as RNA polymerase promoter regions are also suitable primer binding sites.

### Transposase

Methods disclosed herein comprise inserting an insertion fragment such as those discussed herein into a nucleic acid sample. A number of approaches to insertion into the sample are compatible with the disclosure herein, including enzymatic insertion, for example through use of a recombinase, an invertase or a transposase.

Some methods herein use a transposase to treat the target nucleic acids. Methods consistent with the disclosure herein incorporate one or a plurality of consistent elements as recited below and herein. Often, an enzyme is selected to so as not to fragment or otherwise damage the target nucleic acids aside from the insertion process. The transposase reaction is often conducted in a minimal amount of time in order to obtain a density of transposition events at one insertion per 500 to 2000 base pairs, or 3000 base pairs, 4000 base pairs, 5000 base pairs or greater than 5000 base pairs. The transposase binds an insertional nucleic acid and catalyzes insertion or movement of the insertional nucleic acid into a target nucleic acid (e.g. genomic DNA). Some exemplary transposases have DNase activity. Some of the transposases have RNase activity. The transposase often has integrase activity. A retroviral integrase or an enzyme possessing this activity is consistent with the methods herein. A polynucleotidyl transferase is consistent with the methods herein. The transposase is optionally an Escherichia bacterial transposase. The transposase is optionally a Shewanella bacteria transposase. An exemplary transposase is Tn5 transposase. Some transposase examples demonstrate at least 10%, 15%, 20%, 25%, about 30%, 35%, 40%, 45%, 50%, 55%, 60%, 65%, 70%, 75%, 80%, 85%, 90%, 95% or 99% or greater sequence identity to a Tn5 transposase. Some transposases comprises at least one mutation relative to Tn5, such as a mutation that increases the catalytic activity of the transposase. Some exemplary mutations include L372P, using standard one letter nucleic acid abbreviations. Some transposases comprise a DDE motif, understood cases to comprise an aspartate at amino acid 97, an aspartate at amino acid 188 and a glutamate at amino acid 326 in standard numberings of transposases such as Tn5. Alternately, the DDE motif is mutated to an EED motif (e.g. the aspartates are mutated to glutamates and the glutamate is mutated to aspartate at amino acids 97, 188 and 326, respectively). Alternately, the transposase comprises a DDD motif, or a DEE motif, or an EEE motif. A compatible transposase is a Tc1/mariner-type transposase. Another compatible transposase is a sleeping beauty transposase, such as the sleeping beauty transposase is SB100X. Alternately or in combination, the transposase demonstrates at least 10%, 15%, 20%, 25%, about 30%, 35%, 40%, 45%, 50%, 55%, 60%, 65%, 70%, 75%, 80%, 85%, 90%, 95% or 99% or greater sequence identity to SB100X sleeping beauty transposase.

Alternative approaches to sample insertion are consistent with the methods herein. In some cases an important feature of the insertion approach is that it preserves phase information of the nucleic acid molecules into which the inserts are introduced. Although transposase treatment accomplishes this goal, other approaches are similarly contemplated, and any number of approaches that effect insertion at random or otherwise at the desired densities, in some cases without impacting nucleic acid phase information, are contemplated herein and are consistent with the libraries presented herein.

In some alternatives, promoter sequences can be inserted using for example, a nuclease that chews back at a nicked site exposing single stranded genomic DNA. Then a T7 promoter sequence with a random primer can be hybridized. The chewed back portion can then be extended and ligated to the 5′ end of T7 promoter. Alternatively, long molecules (10-100 kb) of genomic DNA can be ligated to T7 promoter sequences to effectively introduce the promoter sites randomly within the genome.

### Other Reagents for Insertion Reaction

The methods disclosed herein comprise combining the integrase such as a transposase, the insertional nucleic acid(s) and target nucleic acid(s) in a reaction buffer. Buffers consistent with the disclosure herein often comprises magnesium and are devoid of sodium diethyl sulfate (SDS), as SDS may denature the integrase (e.g. transposase). A number of transposase reaction conditions are known in the art, and the disclosure herein is consistent with a plurality of variations on reaction conditions.

### Ratios of Transposase Enzyme to Insertional Nucleic Acids & Target Nucleic Acids

A number of ratios are contemplated herein. Generally, reagents are selected to effect local insertion densities of about 500 bp to 2 kb within a sample nucleic acid molecule. Often reagents are selected to effect global insertion densities of about 500 bp to 2 kb within a sample nucleic acid molecule, or within each nucleic acid molecule in a sample. Reagents are optionally selected to effect local or global insertion densities of about 500 bp to 5 kb. Often, reagents are selected to effect local or global insertion densities of about 400 bp, 500 bp, 600 bp, 700 bp, 800 bp, 900 bp, 1 kb, 1.2 kb, 1.4 kb, 1.5 kb, 1.7 kb, 1.8 kb, 2 kb, 2.5 kb, 3 kb, 3.5 kb, 4 kb, 4.5 kb, 5 kb, 5.5 kb, 6 kb, 6.5 kb, 7 kb, 8 kb, 9 kb, or 10 kb within a sample nucleic acid molecule or within each nucleic acid molecule in a sample. An insertion density is sometimes chosen based on the length of RNA transcript produced by an RNA polymerase. An average RNA transcript may have a length of about 2 kb to 10 kb of nucleotides, or a mean of about, 5-8 kb but may range between 1000 and 20,000 nucleotides in length. An insertion density is chosen to have sufficient overlap to allow sequences to be assembled into contigs. Optionally, density is selected to minimize the overlap between RNA transcripts so as to minimize sequence redundancy. Generally, insertion densities of substantially more dense than one insertion per 500 bp result in substantial redundancy of sequence information obtained.

### B. Diluting Multi-Insert Nucleic Acids

Some methods disclosed herein comprise diluting multi-insert nucleic acids into a plurality of containers, to produce a first plurality of diluted multi-insert nucleic acids in a first container and a second plurality of diluted multi-insert nucleic acids in a second container.

The extent of dilution varies according to the eventual objectives for the library. If the library is intended to be used for phase determination, then there are benefits in minimizing the chance that two parental chromosomes will be partitioned into the same compartment. Having two parental chromosomes in a single partition does not preclude phase determination, but the data from these co-diluted chromosomes may be contradictory, and may be excluded from the final analysis.

When the library is intended for de novo genome assembly, the differential segregation of parental chromosomes is not necessarily a priority. In these cases, a priority is often to minimize the amount of DNA per barcode so as to facilitate sequence assembler activity. Co-segregation of two parental chromosomes into a de novo genome assembly may lead to a branched assembly, but one that is likely to be resolved using barcoded reads from other dilutions.

Accordingly, some methods comprise diluting multi-insert nucleic acids, wherein the multi-insert nucleic acids comprise genomic DNA. Some methods comprise diluting the multi-insert nucleic acids comprising genomic DNA into a plurality of containers, such that no two containers contain the same chromosome. Some methods comprise diluting the multi-insert nucleic acids comprising genomic DNA into a plurality of containers, such that no two containers contain identical samples. Some methods comprise diluting the multi-insert nucleic acids comprising genomic DNA into a plurality of containers, such that the likelihood that two containers contain the same genomic sample sequence is very low. Some methods comprise diluting the multi-insert nucleic acids comprising genomic DNA into a plurality of containers, such that the likelihood that two containers contain the same haplotype is a percentage selected from less than about 1%, less than about 2%, less than about 3%, less than about 4%, less than about 5%, less than about 6%, less than about 7%, less than about 8%, less than about 9%, less than about 10%. Some methods comprise diluting the multi-insert nucleic acids comprising genomic DNA into a plurality of containers, such that the likelihood that two containers contain the same haplotype or sub-haploid fraction is a percentage selected from less than about 5%, less than about 10%, less than about 15%, less than about 20%, less than about 25%, less than about 30%, less than about 35%, less than about 40%, less than about 45% and less than about 50%.

Some methods comprise diluting the multi-insert nucleic acids comprising genomic DNA into a plurality of containers, such that the haplotype frequency in a container is very low. The haplotype frequency in a container is very low if there is less than about 10, less than about 5, less than about 4, less than about 3, less than about 2 or less than about 1 copy of a haplotype in each container of the plurality of containers.

Some methods comprise diluting multi-insert nucleic acids into a plurality of containers, such that each container of the plurality of containers contains less than about 1000, less than about 500, less than about 200, less than about 100, less than about 50, less than about 20 or less than about 10 multi-insert nucleic acids. Some methods comprise diluting multi-insert nucleic acids into a plurality of containers, such that each container of the plurality of containers does not contain more than one multi-insert nucleic acid.

### Containers

Some methods disclosed herein comprise diluting multi-insert nucleic acids into a plurality of containers. The plurality of containers may comprise a container selected from a tube, a well, a microwell, a nanowell, droplet, a microdroplet, or an otherwise spatially separated compartment.

Methods disclosed herein comprise conducting the reaction which inserts the insertional nucleic acid, or “transposon,” into the target nucleic acid, for example a transposase reaction, in the same container as the amplification of the template, for example by in vitro transcription. Often, this “one pot reaction” has the benefit of minimizing the manipulation steps of the sample and target nucleic acid which maintains the structural integrity of the sample target nucleic acid.

The container is often part of a solid support. The solid support may be selected from a rack, a chip, a column, a slide, a wafer, and a bead. Optionally, the bead comprises streptavadin. Some methods further comprise incorporating a biotin molecule into the multi-insert nucleic acids.

The container is optionally a plate. An example is a microplate, having a container that is a microwell. Often, the microplate comprises about 96 microwells, about 384 microwells, about 1536 microwells, about 3456, or about 9600 wells.

Containers may have a volume of 1 L, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL, 21 μL, 22 μL, 23 μL, 24 μL, 25 μL, 26 μL, 27 μL, 28 μL, 29 μL, or 30 μL. Often, the containers have a volume of 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL or 100 μL. Alternately, the volume of the container is about 2 μL. The volume of the container is about 55 μL in other cases. Alternately, the volume of the container is about 330 μL. Some container volumes are on a nanoliter scale. Alternately, some container volumes are on a picoliter scale.

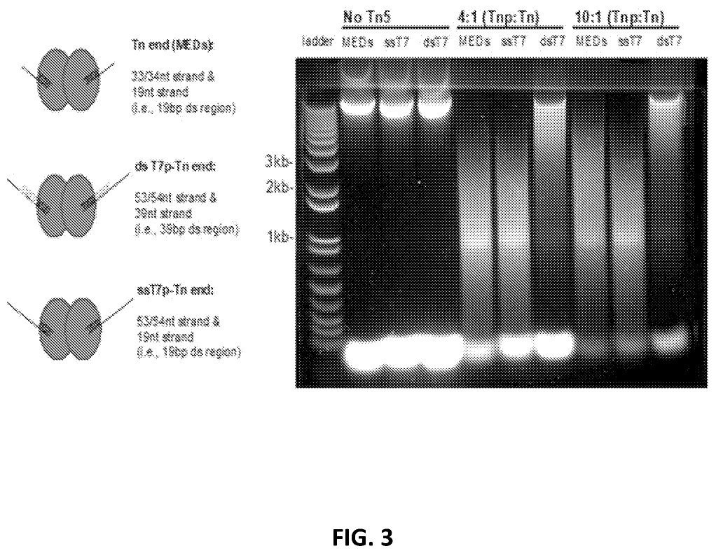

In some embodiments, a Tn5 transposase is used. In some embodiments a high concentration of the transposase is used. In some embodiments the transposon inserts the synthetic nucleic acid at 1000 nucleotides intervals, thereby generating nucleic acid fragments a little over 1000 nucleotides. A successful run of the insertion of T7 promoter in human genomic DNA sample is demonstrated in FIG. 3, where insertion at an interval of 1000 bp generates DNA fragmentation as evidenced in gel electrophoresis. Samples lacking transposase did not generate the fragmentation.

### C. Washing

Some methods disclosed herein comprise washing the multi-insert nucleic acids to remove excess insertional nucleic acids and/or the integrase (e.g. transposase) after introducing the insertional nucleic acids into the target nucleic acid(s) to produce the plurality of multi-insert nucleic acids. Washing optionally occurs before diluting the plurality of multi-insert nucleic acids into a plurality of containers, or may occur after diluting the plurality of multi-insert nucleic acids into a plurality of containers.

Optionally, intermediate libraries are treated to remove the original nucleic acids prior to reverse transcription. This is accomplished through DNase treatment of the sample comprising the amplified RNA intermediate library and the original DNA, followed by, for example, heat inactivation of the DNase activity.

### D. RNA Polymerase-Driven RNA Synthesis From Multi-Insert Nucleic Acids

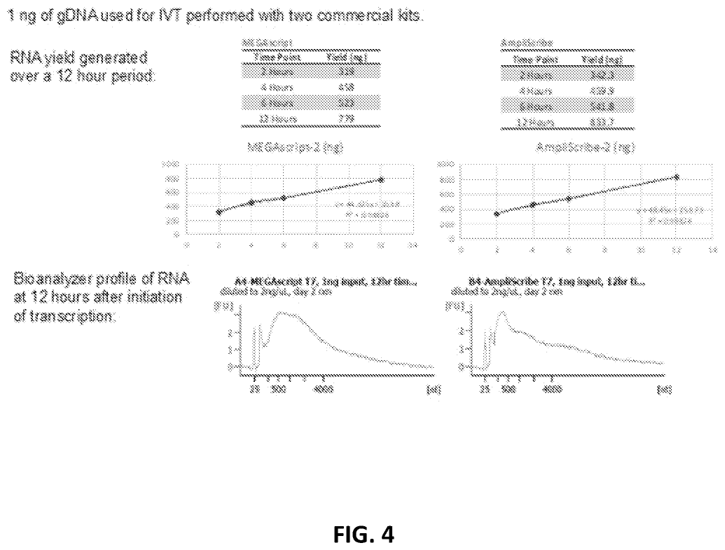

The multi-insert nucleic acids generated using the methods described in the preceding section are subjected to in vitro RNA transcription from the inserted promoter site. For example the inserted T7 promoter serves as the promoter for T7 polymerase. In vitro transcription from T7 promoter is achieved using the standard protocols. An RNA library is thereby generated comprising the insertion sequences and the sequence from the original sequence from the isolated sample. Amplification is achieved through the random insertion of a promoter sequence throughout a sample, followed by transcription-based generation of an intermediate RNA library. FIG. 4 demonstrates results from an in vitro RNA generation as described herein, using T7 promoter, using two different commercially available kits. T7 promoters were introduced into the genomic DNA using transposon. In each case a starting material of 1 ng of genomic DNA was used. The RNA yield was measured at the time intervals indicated, 2 h, 4 h, 6 h and 12 h in each of the two runs. Bioanalyzer profile of the RNA at 12 h is shown.

The intermediate RNA library consists of members that are derived directly from the sample template rather than having their synthesis be directed from prior synthesis intermediates as is the case in PCR based, phi-29 based, or other amplification methods. As a result, errors that may be introduced in the synthesis of a particular library intermediate RNA molecule are not amplified and do not propagate throughout the process of library generation. Intermediates are synthesized directly from the sample, and thus any errors introduced in a library constituent are likely to be unique to that molecule. Thus, independent of the overall error rate in library synthesis, errors in synthesis are likely to be unique or at least very rare in the intermediate library. As a result, they are easily distinguished from mutations present in the sample, even rare mutations, in downstream sequence analysis.

Another benefit of the library's RNA intermediates is that, unlike PCR or DNA synthesis-based library amplification, the 3′ ends of RNA intermediates do not serve as primers for further synthesis. Thus, chimeric molecule formation is dramatically reduced in library generation relative to methods relying on PCR or phi-29.

### E. Generation of Oligonucleotide Probes

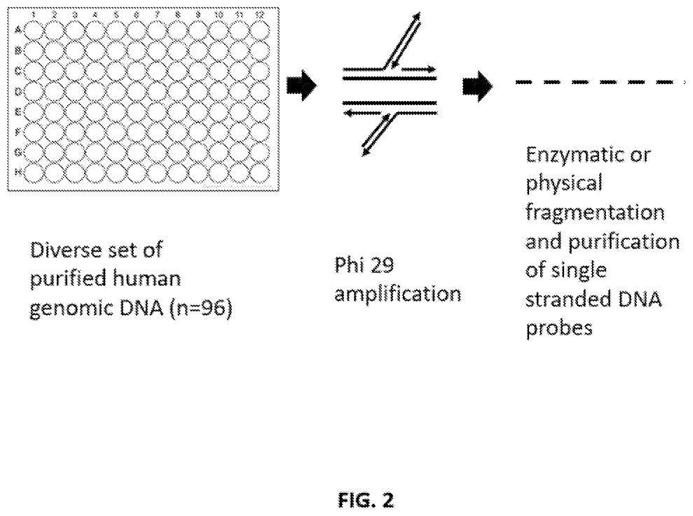

Unwanted nucleic acid sequences such as nucleic acid sequences from a host species or a contaminant species can be generated by fragmenting genomic DNA from the species into oligonucleotide fragments comprising 20, 30, 40, 50, 60, 70, 80, 90, 100, 200, 300, 400, 500 nucleotides in length, using nuclease digestion. In some embodiments the process involves first amplification of genomic DNA, e.g. using Phi 29 mediated amplification, followed by fragmentation to generate probes (FIG. 2).

In some embodiments, fragmentation can be achieved by mechanical shearing. In some embodiments, fragmentation can be achieved by utilizing focused acoustic shearing. In some embodiments, fragmentation of the genomic DNA can be achieved by sonication. Several sonication devices for the purpose are known in the art and are used in next generation sequencing processes.

In some embodiments, fragmentation can be achieved by hydrodynamic shearing. In some embodiments, mechanical shearing is obtained by passing genomic DNA solution through nebulizer. A nebulizer forces the DNA solution with compressed air to pass through a small hole, thereby causing shearing. Similarly, hydrodynamic shear forces can be generated by pushing DNA through a syringe. Size is controlled by altering the speed at which the DNA is pushed through the syringe. Centrifugation can also be used to create hydrodynamic force, by pulling the DNA sample through a hole with a defined size. The rate of centrifugation determines the degree of DNA fragmentation. DNA fragments generated with hydrodynamic shear forces are typically in the range of 1-75 kb, but require large DNA input amounts (>1 μg) and throughput is low.

In some embodiments, fragmentation can be achieved by enzymatic processes instead of mechanical shearing. Enzymes are available that generate cuts in single or double strands, depending on the enzyme, and at random intervals, or at specific intervals, thereby allowing controlled fragment size. For example, caspase activated nuclease in a viable cell can generate fragmented genomic DNA. S1 endonuclease generates cleavage on single stranded DNA. Other examples of such nuclease include micrococcal nucleases, certain endonucleases. In some embodiments, DNA is chemically modified to render susceptibility to certain enzymes. In other embodiments, programmable nucleases are utilized. Genome editing tools include meganucleases (MNs), zinc finger nucleases (ZFNs), transcription activator-like effector nucleases (TALENs), clustered regularly interspaced short palindromic repeat (CRISPR)-associated nuclease Cas9, and targetrons (T. K. Guha et al./Computational and Structural Biotechnology Journal 15 (2017) 146-160). All of them can achieve precise genetic modifications by inducing targeted DNA double-strand breaks (DSBs).

Other methods include synthetic methods of generating short length whole genome amplification methods. In some embodiments, random primer mediated amplification of oligonucleotide sequences from the whole genome can be used to generate probes.

Probes generated for the purpose of the instant disclosure are DNA probes. The probes can be tagged or barcoded. Probes are single stranded or rendered single stranded before using in the hybridization procedure.

For the purpose of the instant disclosure, the probes generated for the process are often referred to as oligonucleotide probes. The oligonucleotide probes referred to here can be 10, 20, 30, 40, 50, 60, 70, 80, 90, 100, 200, 300, 400, 500, 600 or more nucleotides in length and any length in between.

Unwanted nucleic acid sequences can be specific sequences in the genome. Such sequences could be for example, highly repetitive sequences, ribosome DNA sequences, LINE or SINE sequences, mitochondrial DNA sequences, heterochromosomal sequences. In some embodiments, specific and/or unique sequences that are considered unwanted sequences for the purpose of the application, can be generated into specific probes by first generating RNA guide to then generate specific RNA sequence-guided double stranded breaks using CRISPR-Cas system. In some embodiments, specific restriction endonuclease sites are inserted in the desired regions flanking the sequences comprising the unwanted sequences, which are then later contacted with the respective restriction endonucleases to generate cuts at the specific endonuclease sites.

### F. Hybridization and RNase H Digestion

The methods describe herein comprise (i) generation of RNA from a nucleic acid from a sample from a promoter that is inserted at intervals within the nucleic acid, and the RNA strands generated comprises one or more adapter sequences as described in the preceding section; and (ii) generation of probe sequences from unwanted nucleic acid, wherein the probe sequences are DNA sequences, as described above. Following the above, the generation of (i) and (ii), the RNA and the probes are mixed and hybridized. In some embodiments, the mixed sequences are subjected to elevated temperatures for about 1, 2, 3, 4, 5,6, 7, 8, 9,10, 20, 30, 40, 50 or 60 minutes for generating single stranded molecules, followed by lowering temperature allowing hybridization for about 10, 20, 30, 40, 50, 60, 70, 80, 90, 100, 110 or 120 minutes or more. In some embodiments, the mixed nucleic acids are subjected to 50° C., 55° C., 60° C., 65° C., 70° C., 75° C., 80° C., 85° C., or 90° C. or 100° C., for about 5 minutes for strand separation. In some embodiments, the mixed nucleic acids are subjected to 50° C., 55° C., 60° C., 65° C., 70° C., 75° C., 80° C., 85° C., or 90° C. or 100° C., for about 10 minutes for strand separation. In some embodiments, the mixed nucleic acids are subjected to 50° C., 55° C., 60° C., 65° C., 70° C., 75° C., 80° C., 85° C., or 90° C. or 100° C., for about 5 minutes for strand separation. In some embodiments, the mixed nucleic acids are subjected to 50° C., 55° C., 60° C., 65° C., 70° C., 75° C., 80° C., 85° C., or 90° C. or 100° C., for about 15 minutes for strand separation. In some embodiments, the mixed nucleic acids are subjected to 50° C., 55° C., 60° C., 65° C., 70° C., 75° C., 80° C., 85° C., or 90° C. or 100° C., for about 20 minutes for strand separation. In some embodiments, the mixed nucleic acids are subjected to 50° C., 55° C., 60° C., 65° C., 70° C., 75° C., 80° C., 85° C., or 90° C. or 100° C., for about 25 minutes for strand separation. In some embodiments, the mixed nucleic acids are subjected to 50° C., 55° C., 60° C., 65° C., 70° C., 75° C., 80° C., 85° C., or 90° C. or 100° C., for about 30 minutes for strand separation. In some embodiments, the mixed nucleic acids are subjected to an elevated temperature of about 50° C. for 20 minutes to 60 minutes for achieving strand separation. In some embodiments, the mixed nucleic acids are subjected to about 60° C. for 10 minutes to 50 minutes for achieving strand separation. In some embodiments, the mixed nucleic acids are subjected to about 65° C. for 10 minutes to 60 minutes for achieving strand separation. In some embodiments, the mixed nucleic acids are subjected to about 70° C. for 10 minutes to 30 minutes for achieving strand separation. In some embodiments, the mixed nucleic acids are subjected to about 75° C. for 5 minutes to 30 minutes for achieving strand separation. In some embodiments, the mixed nucleic acids are subjected to about 80° C. for 5 minutes to 30 minutes for achieving strand separation. In some embodiments, the mixed nucleic acids are subjected to 90° C. for 2 minutes to 20 minutes for achieving strand separation. In some embodiments, the temperature is then cooled to about 50° C., and incubated for 10, 20, 30, 40, 50 or 60 minutes. In some embodiments, the temperature is then cooled to about 45° C., and incubated for 10, 20, 30, 40, 50 or 60 minutes. In some embodiments, the temperature is then cooled from about 90° C. to about 60° C., and incubated for 10, 20, 30, 40, 50 or 60 minutes.

The reactions are carried out in presence of adequate buffers, usually available commercially but also can be generated in-house.

Following hybridization, the solution comprising hybridized and unhybridized sequences are subjected to treatment with RNase H. Typically, RNase H reaction is carried out by incubating the nucleic acid in presence of a suitable concentration of the enzyme in a suitable buffer (commercially available) in a nuclease free aqueous solution at 37° C. for 20-30 minutes. The reaction is terminated using 25 mM EDTA. Depending on the size and nucleic acid concentration and volume of RNA:DNA hybridization mix, the RNase reaction is suitably titrated to obtain optimum digestion.

The digested products are cleaned by nucleic acid purification kits, to remove the digested products, optionally obtaining purified RNA that is not digested by appropriate washing and centrifugation using size exclusion filters. The washing and purification steps can include elimination of DNA as contaminant in the RNA recovery procedure.

The undigested RNA is then collected or harvested in a suitable volume for downstream applications, e.g. sequencing, amplification and library generation (FIG. 1A).

### Sequencing Target Nucleic Acids

Some methods disclosed herein comprise sequencing the multi-insert nucleic acids and/or multi-insert nucleic acid fragments. A number of sequencing methods are known to one of skill in the art. Methods of sequencing nucleic acids disclosed herein also include methods described in PCT application number PCT/US2015/049249 filed Sep. 9, 2015 which is hereby incorporated by reference in its entirety.

Some methods further comprise pooling amplified target nucleic acids or amplified target nucleic acid fragments from two more containers before sequencing. Some methods further comprise pooling the amplified multi-insert nucleic acids or amplified multi-insert nucleic acid fragments from two more containers before sequencing. The sequencing may read the tag of an amplified multi-insert nucleic acid/target nucleic acid, thereby identifying the container from which it was pooled.

Some methods comprise annealing an oligonucleotide required for sequencing to the multi-insert nucleic acids or multi-insert nucleic acid fragments. Some sequencing comprises ligating an oligonucleotide required for sequencing to the multi-insert nucleic acids or multi-insert nucleic acid fragments. Some methods comprise utilizing the adapter sequence or portion thereof to sequence the multi-insert nucleic acids or multi-insert nucleic acid fragments.

Methods of nucleic acid sequencing are well-known and described thoroughly in the art. The methods disclosed herein may comprise any standard or known method of sequencing.

Determination of the sequence of an amplified nucleic acid may be performed using a sequencing method selected from a variety of sequencing methods including, but not limited to, ion detection technology, DNA nanoball technology, nanopore-based sequencing technology, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads, wobble sequencing, multiplex sequencing, polymerized colony (POLONY) sequencing; nanogrid rolling circle sequencing (ROLONY), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods such as cyclic array sequencing using platforms such as Roche 454, Illumina Solexa, ABI-SOLiD, ION Torrents, Complete Genomics, Pacific Bioscience, Helicos, Polonator platforms, are consistent with the disclosure herein.

Determination of the sequence of an amplified nucleic acid performed by a next-generation sequencing (NGS) method is consistent with the disclosure herein. NGS applies to genome sequencing, genome resequencing, transcriptome profiling (RNA-Seq), DNA-protein interactions (ChIP-sequencing), and epigenome characterization. Some methods disclosed herein comprise an NGS method selected from, but are not limited to, massively parallel signature sequencing (MPSS), Polony sequencing, 454 pyrosequencing, Ion Torrent semiconductor sequencing, Heliscope single molecule sequencing, single molecule real time (SMRT) sequencing and microfluidic Sanger sequencing.

### Linking Sequences Together

Some methods disclosed herein further comprise determining the phase of resulting sequences. Methods may comprise aligning a first sequence and second sequence according to an overlapping sequence common to the first sequence and the second sequence.

Often, the multi-insert nucleic acids are diluted into a container before amplification/sequencing, such that the likelihood of sequencing two different haplotypes or two different alleles from a single partition or container is very small.

Often, the multi-insert nucleic acids are diluted into a container before amplification/sequencing, and more than one copy of a haplotype or allele are diluted into a first container. The more than one copy may originate from a same chromosome, or may originate from a different chromosome.

### Sequencing Low Complexity and Repetitive Nucleic Acids

A benefit of methods and libraries disclosed herein is that they facilitate sequencing of genomic nucleic acid samples, including samples having locally or globally repetitive regions. That is, regions comprising a sequence unit that is completely or incompletely repeated at a single locus or at multiple discrete loci are accurately sequenced using methods or compositions as disclosed herein.

Current methods of sequencing are unable to accurately provide a sequence in such nucleic acid regions because, some regions comprise a repetitive sequence and such nucleic acid sequences are difficult to assemble. Most alternative approaches may identify repetitive regions, but do not accurately establish the number of or sequence of the repeats of a given locus, and often are unable to assign a point mutation to one rather than another monomer of a repeat region or to a repeat region at one or another locus.

Though the practice of the methods herein, an insertion nucleic acid fragment is introduced at positions distributed throughout a nucleic acid sample, such that inserts are distributed at a density of about 1 every 500 pb to 2 kb, 3 kb, 4 kb 5 kb or greater than 5 kb. Importantly, insertion site determination is largely or completely independent of sample nucleic acid sequence, such that the insertion distribution patter is independent of the underlying sample nucleic acid sequence. As a result regions of a sample that are repetitive and therefore difficult to sequence at a given locus, or difficult to assign to one or another repetitive locus of a genome, are provided with an overlying ‘barcode’ or ‘insertion fingerprint’ of insertion fragment sequence, such that the combination of underlying sample sequence and inserted sequence such as ME borders and RNA polymerase primer sequence is no longer repetitive.

Advantages provided by methods described herein include providing a fingerprint by inserting an insertional nucleic acid into the sample nucleic acid comprising a target nucleic acid. This insertion occurs in several locations across the region, providing a common locus by which to place sequence reads when assembling the sequence data.

Accordingly, sequence reads that would otherwise map to multiple loci or map only to a single, highly overrepresented repeat monomer, are mapped according to their start position and 5′ insert sequence to a specific insertion site within a repetitive region. Furthermore, RNA transcription products directed by adjacent insertion events will often span an insert in a repetitive region, such that insertion site sequences in their local sequence context are obtained through sequencing of a library as generated herein. Accordingly, repeat region sequence is mapped according to its sequence start site and according to insertion sites as indicated by additional RNA intermediate reads, such that repeat region sequences are mapped to their locus in a nucleic acid sample sequence such as a genomic sample, rather than mapping to, for example, a single, highly covered but poorly assembled monomer of a repeat sequence.

Thus, repetitive sequence of a nucleic acid sample is sequenced by inserting a plurality of nucleic acid inserts into nucleic acid encoding the repetitive sequence, generating library constituents anchored by the plurality of nucleic acid inserts, sequencing the library constituents and assembling sequences of the library constituents such that sequence reads having repetitive region sequence and insert sequence having common junctions are assembled to common loci. The nucleic acid inserts in many cases comprise RNA polymerase promoter sequence, such that they direct synthesis of a population of RNA molecules spanning the promoter sequence, adjacent ME sequence, and insertion-adjacent repetitive nucleic acid sequence. RNA molecules synthesized hereby comprise insertion sequence, insertion adjacent repetitive sequence and often span at least one adjacent insertion site, and in some cases span at least one repetitive junction to nonrepetitive genomic or other sample sequence. Accordingly, regions that are otherwise partially or completely composed of repetitive nucleic acid sequence are rendered nonrepetitive through the introduction if insertion sequence at random in a plurality of repeated monomer sequences in a nucleic acid sample.

What is claimed is:

1. A method of selective depletion of non-target nucleic acid sequences from a sample wherein the sample comprises a first plurality of nucleic acid molecules comprising target nucleic acid sequence and a second plurality of nucleic acid molecules comprising non-target nucleic acid sequences, the method comprising:
(a) generating RNA molecules from fragments generated from the first and second plurality of nucleic acid molecules, each fragment comprising an insert comprising a promoter sequence for an RNA polymerase, wherein the sum of the RNA molecules generated from the fragments comprise sequences from the first and second plurality of nucleic acid molecules;
(b) generating DNA probes comprising the non-target nucleic acid sequences;
(c) hybridizing RNA molecules from (a) and DNA probes from (b) under conditions suitable for generating RNA:DNA hybrid molecules;
(d) subjecting the RNA:DNA hybrid molecules from (c) to RNase H treatment, thereby selectively depleting the RNA in the RNA:DNA hybrid molecules; and recovering unhybridized RNA comprising the target nucleic acid sequence, selectively depleted of the non-target nucleic acid sequence.

2. A method of detecting the presence or absence of a target nucleic acid from a sample comprising a first plurality of nucleic acid molecules comprising target nucleic acid sequences and a second plurality of nucleic acid molecules comprising non-target nucleic acid sequences, the method comprising:
depleting the second plurality of nucleic acid molecules comprising non-target nucleic acid sequences from the first plurality of nucleic acid molecules by selective hybridization of RNA comprising the non-target nucleic acid sequences with single stranded DNA oligonucleotide molecules comprising the non-target nucleic acid sequences under conditions suitable for generating RNA:DNA hybrid molecules; treating the RNA:DNA hybrid molecules with RNase H thereby digesting the RNA in the RNA:DNA hybrid molecules; and collecting the resulting undigested RNA;
detecting the presence or absence of a target nucleic acid sequence in the resulting undigested RNA or in a DNA derived therefrom.

3. A method of enriching a target nucleic acid from a sample comprising a first plurality of nucleic acid molecules comprising a target nucleic acid sequence and a second plurality of nucleic acid molecules comprising a non-target nucleic acid sequences, the method comprising:
depleting the second plurality of nucleic acid molecules comprising the non-target nucleic acid sequences from the second plurality of nucleic acid molecules by selective hybridization of RNA comprising the non-target nucleic acid sequences with single stranded DNA oligonucleotide molecules comprising the non-target nucleic acid sequences under conditions suitable for generating RNA:DNA hybrid molecules; treating the RNA:DNA hybrid molecules with RNase H thereby digesting the RNA in the RNA:DNA hybrid molecules; and collecting the resulting undigested RNA;
enriching the target nucleic acid in the resulting undigested RNA or in a DNA derived therefrom.

4. The method of any one of claims 1-3, further forming a nucleic acid library comprising the target nucleic acid sequence.

5. The method of any one of claims 2-3, wherein the RNA comprising the target nucleic acid is generated by in vitro transcription of the plurality of nucleic acid molecules comprising target nucleic acid sequence and non-target nucleic acid sequences.

6. The method of any one of the claims 1-5, wherein prior to generating the RNA, the plurality of nucleic acid molecules comprising target nucleic acid sequence and non-target nucleic acid sequences are fragmented by insertion of an RNA polymerase promoter sequence at intervals within the plurality of nucleic acid molecules, such that each fragment comprises an RNA polymerase promoter upstream of the sequence.

7. The method of claim 6, wherein the RNA polymerase promoter sequence comprises a sequence from a promoter selected from the list of promoters consisting of a T7, T3, T7lac, SP6, pL, CMV, SV40, CaMV35S, araBAD, trp, lac, Ptac, pol I, pol II, pol III, EF1a, PGK1, Ubc, beta actin, CAG, TRE, UAS, Ac5, Polyhedrin, CaMKIIa, ALB, GAL1, GAL10, TEF1, GDS, ADH1, Ubi, H1, and U6.

8. The method of claim 6, wherein the promoter sequence is a T7 promoter sequence.

9. The method of claim 6, further comprising insertion of one or more synthetic adaptor nucleic acid sequences or one or more primer sequences.

10. The method of claim 6-9, wherein the insertion is performed by a transposase.

11. The method of claim 10, wherein the transposase is a DNA transposase.

12. The method of claim 10-11, wherein the transposase inserts a first insert sequence at a first insertion site, and a second insert sequence at a second insertion site on the genomic DNA.

13. The method of claim 12, wherein the transposase inserts a first promoter sequence and/or a first adaptor sequence at the first insertion site; and a second promoter sequence and/or a second adaptor sequence at the second insertion site.

14. The method of claim 12 or 13, wherein the transposase inserts a third or subsequent promoter sequence and/or a third or subsequent adaptor sequence at the third or subsequent insertion site.

15. The method of claim 14, wherein the first and the third insertion site is at least 250 nucleotides apart from the first insertion site.

16. The method of any one of claims 12-15, wherein the first insert sequence comprises a restriction endonuclease cleavage site between said first primer binding site and said second primer binding site.

17. The method of any one of claims 1-16, wherein the RNA is generated by in vitro transcription from the inserted RNA polymerase promoter.

18. The method of claim 17, wherein the RNA generated by in vitro transcription results in a high fidelity copy of the plurality of nucleic acid molecules comprising target nucleic acid sequence and non-target nucleic acid sequences.

19. The method of any one of claims 1-18, wherein the RNA generated is amplified from in vitro transcribed RNA.

20. The method of any one of claims 1-19, wherein the sample comprises heterogenous nucleic acid molecules.

21. The method of any one of claims 1-20, wherein the sample comprises genomic DNA of one or more species.

22. The method of claim 21, wherein the sample comprises genomic DNA of one or more different organisms.

23. The method of claim 21, wherein the sample comprises genomic DNA of a microbial species comprising the target nucleic acid sequence, and genomic DNA of a host species comprising the non-target nucleic acid sequences.

24. The method of claim 21, wherein the sample comprises genomic DNA of a host species comprising the target nucleic acid sequence, and genomic DNA of a microbial species comprising the non-target nucleic acid sequences.

25. The method of any one of the claims 1-3, wherein the DNA probe is generated from nucleic acid comprising non-target nucleic acid sequences.

26. The method of any one of the claims 1-3, wherein the DNA probe is synthesized.

27. The method of any one of the claims 1-3, wherein the DNA probe is generated from a sample nucleic acid or a portion thereof, by cleavage to generate oligonucleotide fragments used as probes.

28. The method of claim 25, wherein the DNA probes are oligonucleotide probes that are less than 500 nucleotides long.

29. The method of any one of claims 1-3, wherein the prior to hybridization, the nucleic acids are subjected to denaturing conditions in elevated temperatures of 60° C. or more for at least 10 minutes.

30. The method of any one of claims 1-3, wherein depleting comprise reducing at least by greater than 50% the sequence comprising the non-target nucleic acid sequences compared to the level prior to depletion.

31. A method of selective depletion of non-target ribonucleic acid (RNA) sequences from a sample wherein the sample comprises a first plurality of RNA molecules comprising target RNA sequences and a second plurality of RNA molecules comprising non-target RNA sequences, the method comprising:
(a) obtaining DNA probes comprising non-target nucleic acid sequences;
(b) hybridizing the plurality of RNA molecules with the DNA probes from (a) under conditions suitable for generating RNA:DNA hybrid molecules; and
(c) subjecting the RNA:DNA hybrid molecules from (b) to RNase H treatment, thereby selectively depleting the RNA in the RNA:DNA hybrid; and recovering unhybridized RNA comprising the target nucleic acid sequence, selectively depleted of the non-target nucleic acid sequence.

32. A method of detecting the presence or absence of a target nucleic acid from a sample comprising a first plurality of ribonucleic acid (RNA) molecules comprising target RNA sequences and a second plurality of RNA molecules comprising non-target RNA sequences, the method comprising:
depleting non-target RNA sequences of the second plurality of RNA molecules by selective hybridization of an RNA comprising the non-target RNA with single stranded DNA oligonucleotide molecules having sequence identity to the non-target RNA under conditions suitable for generating RNA:DNA hybrid molecules; treating the RNA:DNA hybrid molecules with RNase H thereby digesting the RNA in the RNA:DNA hybrid molecules; and collecting the resulting undigested RNA;
detecting the presence or absence of a target nucleic acid sequence in the resulting undigested RNA or in a DNA derived therefrom.

33. A method of enriching a target ribonucleic acid (RNA) from a sample comprising a first plurality of RNA molecules comprising target RNA sequence and a second plurality of RNA molecules comprising non-target RNA sequence, the method comprising:
depleting the non-target RNA sequences of the second plurality of RNA molecules from the first plurality of RNA molecules by selective hybridization of an RNA comprising the non-target sequence with a single stranded DNA oligonucleotide molecule having sequence identity with the non-target sequence under conditions suitable for generating RNA:DNA hybrid molecules; treating the RNA:DNA hybrid molecules with RNase H thereby digesting the RNA in the RNA:DNA hybrid molecules; and collecting the resulting undigested RNA;
enriching the target nucleic acid in the resulting undigested RNA or in a DNA derived therefrom.

34. The method of any one of claims 31-33, further forming a nucleic acid library comprising the target nucleic acid sequence.

35. The method of any one of claims 31-34, wherein the sample comprises heterogenous nucleic acid molecules.

36. The method of any one of claims 31-35, wherein the sample comprises RNA derived from one or more species.

37. The method of claim 36, wherein the sample comprises RNA derived from one or more different organisms.

38. The method of claim 36, wherein the sample comprises RNA derived from a microbial species comprising the target nucleic acid sequence, and RNA derived from a host species comprising the non-target nucleic acid sequences.

39. The method of claim 36, wherein the sample comprises RNA derived from a host species comprising the target nucleic acid sequence, and RNA derived from a microbial species comprising the non-target nucleic acid sequences.

40. The method of any one of the claims 31-33, wherein the DNA probe is generated from nucleic acid comprising non-target nucleic acid sequences.

41. The method of any one of the claims 31-33, wherein the DNA probe is synthesized.

42. The method of any one of the claims 31-33, wherein the DNA probe is generated from a sample nucleic acid or a portion thereof, by cleavage to generate oligonucleotide fragments used as probes.

43. The method of claim 42, wherein the DNA probes are oligonucleotide probes that are less than 500 nucleotides long.

44. The method of any one of claims 31-33, wherein the prior to hybridization, the nucleic acids are subjected to denaturing conditions in elevated temperatures of 60° C. or more for at least 10 minutes.

45. The method of any one of claims 31-33, wherein depleting comprise reducing at least by greater than 50% the sequence comprising the non-target nucleic acid sequences compared to the level prior to depletion.

46. A nucleic acid molecule comprising a sequence comprising a target nucleic acid sequence that is enriched from a sample comprising a first plurality of nucleic acid molecules comprising target nucleic acid sequence and a second plurality of nucleic acid molecules comprising non-target nucleic acid sequences using any one of the methods of claims 1-45.
